(19) United States
(12) Patent Application Publication
(10) Pub. No.: US 2025/0322969 A1
(43) Pub. Date: Oct. 16, 2025

(54) SYSTEMS AND METHOD FOR IMPROVING HEALTHY LONGEVITY ("HEALTHSPAN") FIELDS

(71) Applicant: Evity Technologies, Inc., Corte Madera, CA (US)

(72) Inventors: Joseph Brilliant, Corte Madera, CA (US); Richard How-Chi Au, Corte Madera, CA (US); Lawrence Brilliant, Corte Madera, CA (US)

(21) Appl. No.: 19/177,055

(22) Filed: Apr. 11, 2025

Related U.S. Application Data
(60) Provisional application No. 63/633,507, filed on Apr. 12, 2024.
(60) Provisional application No. 63/633,512, filed on Apr. 12, 2024.

(51) Int. Cl. G16H 70/60; G16H 10/60; G16H 40/20; G16H 50/70

(52) U.S. Cl. CPC G16H 70/60; G16H 10/60; G16H 40/20; G16H 50/70

(57) ABSTRACT

Provided herein are systems and methods for improving healthspan. For example, provided herein are systems and methods that generate actionable interventions, based on expert curated artificial intelligence (AI) systems, to increase healthspan.

### CROSS REFERENCE TO RELATED APPLICATIONS

This application claims the benefit of U.S. Provisional Application No. 63/633,507, filed Apr. 12, 2024, and 63/633,512, filed Apr. 12, 2024, the contents of which are herein incorporated by reference in their entirety.

### FIELD

Provided herein are systems and methods for improving healthspan. For example, provided herein are systems and methods that generate actionable interventions, based on expert curated artificial intelligence (AI) systems, to increase healthspan.

### BACKGROUND

Longevity is the achievement of a long life. People hope for longevity to experience many years of quality time with family and friends and to have sufficient time to learn about and explore the world. However, living many years does not necessarily equate to having a healthy and happy longevity, referred to herein as “healthspan.” And “healthspan” which may be one, two or three decades less than “lifespan” is not equally distributed and can vary widely by age, gender, race, zip codes, or census tracts. While many factors associated with healthspan have been carefully studied, such as diet, smoking avoidance, sleep quality, and social connections, there are innumerable individual factors that contribute in complex and interactive ways to well-being and lifespan. What is needed are systems and methods to better understand, prioritize, manage, and take action on the factors that contribute to and achieve both longevity and healthspan.

### SUMMARY

In some embodiments, provided herein are hybrid/human “expert in the loop” AI systems and methods. In some embodiments, the systems and methods include granular attribution and revenue sharing for curation participants. These approaches, alone and/or in combination facilitate the generation of more accurate, trustworthy, actionable, and sustainable decision support guidance.

In some embodiments, provided herein are systems for responding to healthspan queries. In some embodiments, the systems comprise a computer processor configured to: a) receive one or more healthspan queries; b) process said one or more healthspan queries with an artificial intelligence (AI) component that has been trained using expert curated source information from three or more topics selected from atherosclerosis, cancer, neurodegenerative diseases, infectious disease, metabolic syndrome, sarcopenia/orthopedic, violence, lower respiratory disease, despair, maternal morbidity and mortality, menopause, testosterone imbalances, kidney disease, liver disease, accidents and injuries, and geographic place factors, to generate one or more answers; and c) display said one or more answers to a user.

In some embodiments, the AI component has been trained using expert curated source information from five or more of the topics. In some embodiments, the AI component has been trained using expert curated source information from ten or more of the topics. In some embodiments, the AI component has been trained using expert curated source information from each of the topics.

In some embodiments, the three or more topics comprises geographic place factors. In some embodiments, the geographic place factors comprise climate change information.

In some embodiments, the three or more topics comprises despair.

In some embodiments, the expert curated source information is generated using the systems or methods for expert curation of training data source materials for an artificial intelligence (AI) system described herein.

In some embodiments, the user is a health care worker. In some embodiments, the user is a patient.

In some embodiments, provided herein are methods for responding to healthspan queries. In some embodiments, the methods comprise any or all of: receiving one or more healthspan queries, processing the one or more healthspan queries with a system described herein to generate one or more answers, and displaying the one or more answers to a user.

### BRIEF DESCRIPTION OF THE DRAWINGS

FIG. 1A is an exemplary flow chart of integration of a curation system into the administration, modeling, and use of AI systems. FIGS. 1B-1F show workflow details for each of the data sources, curation, model training, administration, and use of the AI systems, respectively.

FIG. 2 is a flow chart of an exemplary curation system workflow.

FIG. 3 is a flow chart of how user generated materials enter into the curation system workflow for AI systems, exemplified with an AI system for health care.

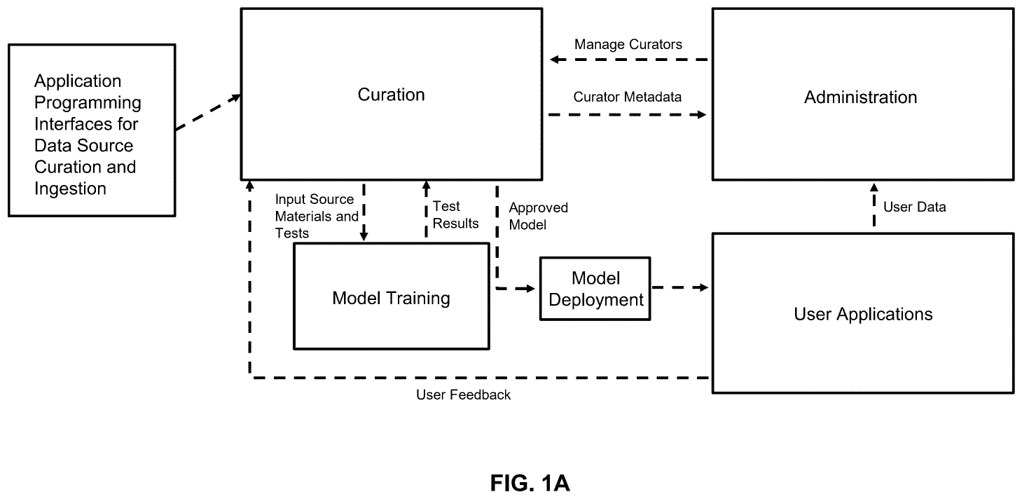

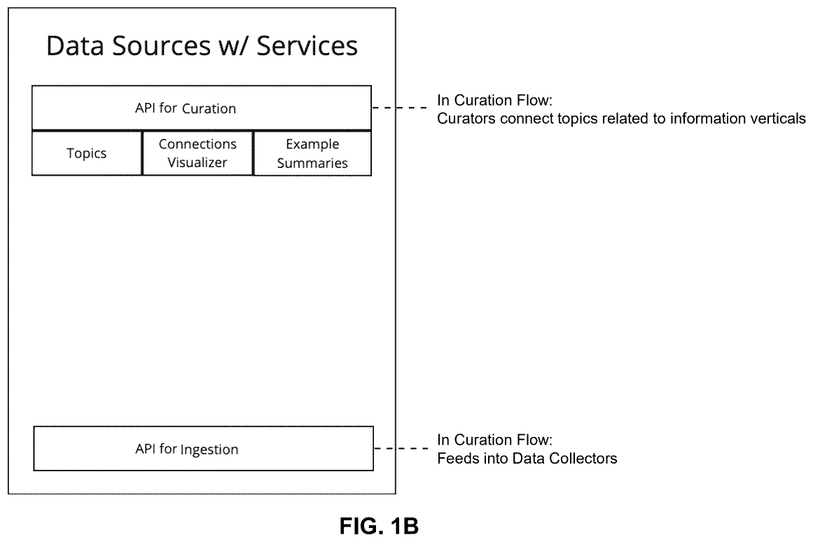

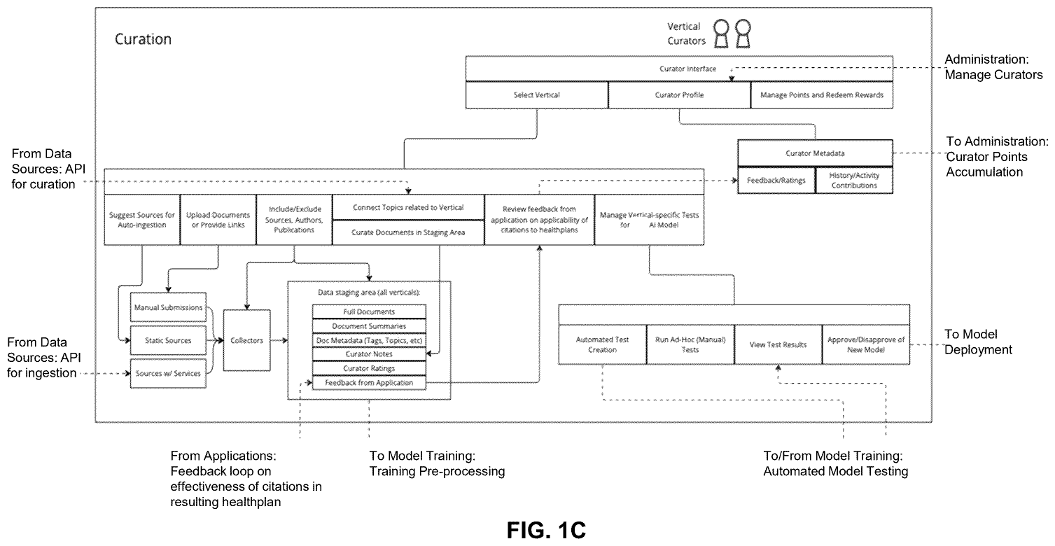

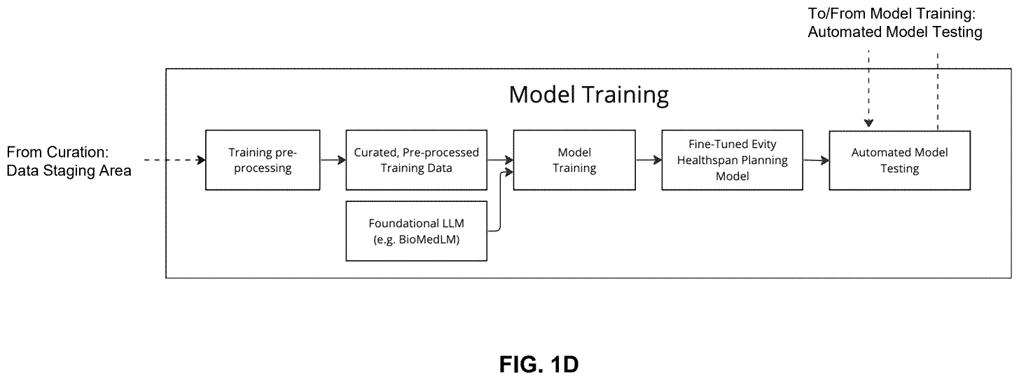

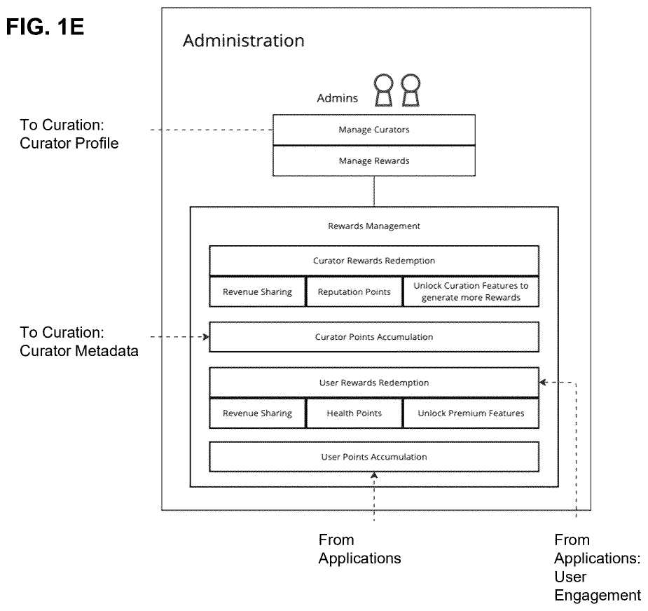

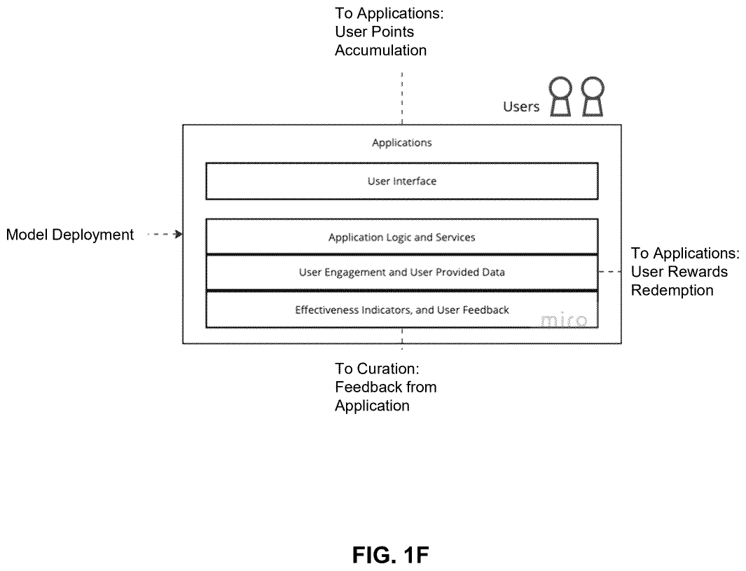

### DEFINITIONS

As used herein, terms and phrases such as “having,” “may have,” “include,” or “may include” a feature (such as a number, function, operation, or component, such as a component) indicate the presence of that feature, and do not preclude the presence of other features. Further, as used herein, the phrase “a or B,” “at least one of a and/or B,” or “one or more of a and/or B” may include all possible combinations of a and B. For example, “a or B,” “at least one of a and B,” and “at least one of a or B” may indicate all of the following: (1) comprises at least one A, (2) comprises at least one B, or (3) comprises at least one A and at least one B. Furthermore, as used herein, the terms “first” and “second” may modify various components without regard to importance, and do not limit the components. These terms are only used to distinguish one component from another. For example, the first user device and the second user device may indicate user devices that are different from each other regardless of the order or importance of the devices. A first component may be termed a second component, and vice-versa, without departing from the scope of the present disclosure.

It will be understood that when an element (such as a first element) is referred to as being (operatively or communicatively) “coupled/coupled” or “connected/connected” to another element (such as a second element), it can be directly coupled or connected/coupled or connected to the other element (such as the second element) or via a third element. Conversely, it will be understood that when an element (such as a first element) is referred to as being “directly coupled”/“directly coupled to” or “directly connected”/“directly connected” to another element (such as a second element), there is no other element (such as a third element) intervening between the element and the other element.

As used herein, the phrase “configured (or set) to” may be used interchangeably with the phrases “adapted to,” “having . . . capability,” “designed to,” “adapted to,” “made to,” or “capable,” as the case may be. The phrase “configured (or set) to” does not substantially mean “specially designed in hardware.” Rather, the phrase “configured to” may indicate that a device is capable of performing an operation with another device or component. For example, the phrase “a processor configured (or arranged) to perform A, B and C” may refer to a general-purpose processor (such as a CPU or an application processor) or a special-purpose processor (such as an embedded processor) that may perform operations by executing one or more software programs stored in a memory device.

The various functions described below may be implemented or supported by one or more computer programs, each formed from computer-readable program code and embodied in a computer-readable medium. The terms “application” and “program” refer to one or more computer programs, software components, sets of instructions, procedures, functions, objects, classes, instances, related data, or a portion thereof adapted for implementation in suitable computer readable program code. The phrase “computer readable program code” includes any type of computer code, including source code, object code, and executable code. The phrase “computer readable medium” includes any type of medium capable of being accessed by a computer, such as Read Only Memory (ROM), Random Access Memory (RAM), a hard disk drive, a Compact Disc (CD), a Digital Video Disc (DVD), or any other type of memory. A “non-transitory” computer-readable medium does not include a wired, wireless, optical, or other communication link that transmits transitory electrical or other signals. Non-transitory computer readable media include media that can permanently store data as well as media that can store data and later rewrite the data, such as rewritable optical disks or erasable memory devices.

Various functions described below may be implemented or supported by one or more natural language communication systems (“NLCS”), which function as networks of interconnected components designed to accept, process, and generate human language. Such systems may include one or more of the following characteristics or structure: input processing, language understanding, knowledge representation, language generation, output presentation, and feedback loops.

NLCS may receive input in the form of text or speech. Inputs not in the form of text, for example, audio, video, images, databases can be converted into text as appropriate. Text input is typically tokenized, while speech input undergoes transcription into textual form through speech recognition algorithms before being tokenized. “Tokenized” refers to the process of segmenting a sequence of text into smaller units, typically words, subwords, or characters, known as tokens. Tokenization involves identifying word boundaries and separating punctuation marks, whitespace, and other delimiters to create a structured representation of the text that can be processed by the NLCS and serves as the basis for further analysis and processing. NLCS may employ various techniques such as statistical models, deep learning architectures, and semantic analysis to understand the meaning of the input text. This includes tasks like named entity recognition, part-of-speech tagging, syntactic parsing, and semantic role labeling to extract relevant information and comprehend the context of the input. Structured databases, knowledge graphs, or embeddings may be utilized to represent information and knowledge extracted from text data.

Inference mechanisms may be used to derive conclusions, make predictions, or answer questions based on the input and various heuristics. This involves various reasoning techniques such as deductive, inductive, or abductive reasoning, as well as probabilistic reasoning to deal with uncertain information. After processing the input and performing any necessary reasoning, NLCS may generate responses or output in natural language form. Generation techniques may include template-based approaches, rule-based systems, or more advanced methods like sequence-to-sequence models with attention mechanisms. The generated output may be presented to the user in a human-readable format, which may involve text rendering for text-based interactions or speech synthesis for voice-based interactions. The generated output may also be presented in non-text based formats e.g., audio, video, images, and the like. Output presentation may also include formatting, summarization, and other post-processing tasks to enhance readability, usability, and relevance. NLCS may also incorporate feedback mechanisms to improve their performance over time. This feedback may come from user interactions, explicit corrections, or implicit signals such as user satisfaction metrics, which may be used to update and refine the system's models and algorithms.

NLCS may include or be supported by a “neural network,” or a computational model consisting of interconnected nodes, or “neurons,” which receive individual input signals, process them, and produce an output signal. Information may flow through the network from an input layer, through hidden layer(s), and then to the output layer. The input layer is the first neuron layer, where input data is fed into the network. Each neuron in the input layer may represent a feature or attribute of the input data. Hidden layers are intermediate layers between the input and output layers in a neural network, which perform transformations on the input data using weighted connections and activation functions. The output layer of a neural network is the final layer, where the network produces its output predictions or classifications. The number of neurons in the output layer may correspond to the number of output classes or dimensions of the prediction. An activation function is a mathematical function applied to the weighted sum of inputs at each neuron in a neural network. Weights and biases are parameters within a neural network that are learned during the training process. Weights may be understood to represent the strength of connections between neurons, determining the influence of one neuron's output on another. Biases are additional parameters added to each neuron that shift the activation function. Neural networks may use various training techniques such as backpropagation. Backpropagation based training may use an algorithm to update the weights of a neural network based on the error between the predicted output and the true output and may involve calculating the gradient of the error with respect to the network's weights and adjusting the weights to minimize the error.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “database” refers to an organized collection of structured information, or data, typically stored electronically in a computer system.

As used herein, the term “knowledge base” refers to a store of information that is available to draw on. When used in reference to curated knowledge bases, the knowledge bases can include not only text, other information contained in curated documents (e.g. in for the form of images, charts, graphs, etc.), or other curated media (e.g., audio, video, images, databases), but also curator annotations that guide when (e.g., for what types of questions) each knowledge base is used to generate responses, and how portions of the knowledge base are used.

The terms and phrases used herein are used only to describe some embodiments of the present disclosure and do not limit the scope of other embodiments of the present disclosure. It is to be understood that the singular includes plural referents unless the context clearly dictates otherwise. All terms and phrases used herein (including technical and scientific terms and phrases) have the same meaning as commonly understood by one of ordinary skill in the art to which embodiments of the present disclosure belong. It will be further understood that terms and phrases, such as those defined in commonly used dictionaries, should be interpreted as having a meaning that is consistent with their meaning in the context of the relevant art and will not be interpreted in an idealized or overly formal sense unless expressly so defined herein. In some instances, the terms and phrases defined herein may be construed to exclude embodiments of the disclosure.

Definitions for other specific words and phrases may be provided throughout this patent document. Those of ordinary skill in the art should understand that in many, if not most instances, such definitions apply to prior, as well as future uses of such defined words and phrases.

None of the description in this application should be read as implying that any particular element, step, or function is an essential element which must be included in the claim scope. The scope of patented subject matter is defined only by the claims. Any other term used in the claims, including, but not limited to, “mechanism,” “module,” “device,” “unit,” “assembly,” “element,” “member,” “apparatus,” “machine,” “system,” “processor,” or “controller,” is understood by the applicants to refer to structures known to those of ordinary skill in the relevant art.

### DETAILED DESCRIPTION

Human-conducted empirical studies have significantly advanced our understanding of the world over the previous centuries, decades, and years. But progress is slow and is often narrowly focused on specific sub-parameters of a specific problem or on simple correlations between a limited number of variables. Artificial intelligence (AI) and machine learning (ML) offer the promise of faster results and the recognition of previously unappreciated complex correlations between variables. Recent years have seen significant achievements in AI/ML, and a huge rise in generative AI and its applications. However, AI/ML systems are limited by the quality of information used to train them. This is particularly evident in applications that generate output containing facts. AI systems with gaps or inaccuracies in their training data may notoriously “hallucinate,” or fail to produce output at all. Depending on the application, mistakes or hallucinations may be harmless and/or manageable. In other applications, hallucinations can have dire consequences. Other issues include problems with attribution, safety, and bias, in addition to the misinformation/hallucinations (see e.g., Menz et al., Current safeguards, risk mitigation, and transparency measures of large language models against the generation of health disinformation: repeated cross sectional analysis, BMJ, 2024, 384: e078538; Tyson and Kennedy, Many Americans think generative AI programs should credit the sources they rely on, Pew Research Center, Mar. 26, 2024; and Editorial, How to support the transition to AI-powered healthcare, Nature Medicine, 30:609-610 (2024); each of which is herein incorporated by reference in its entirety).

Several recent stories have highlighted the problem of hallucinations. A Dec. 3, 2022, post on the Hacker News forum highlighted a hallucination by ChatGPT. The user queried ChatGPT to “provide references that deal with the mathematical properties of lists.” ChatGPT returned five citations by title, author, and hyperlink. The user was “pretty surprised and happy” because searches using the GOOGLE search engine had failed to produce any useful results. It turned out that everyone one of ChatGPT's citations was made up. The references did not exist, and the links were not real. The cited authors never published papers with the recited titles. In this instance, time was wasted and perhaps trust was lost. But there were no dire consequences.

U.S. News reported a story on Jun. 22, 2023, explaining that a federal judge imposed a $5000 fine on two lawyers and a law firm based on submission of legal documents containing fictitious legal citations created by ChatGPT.

A study entitled “Medical Hallucination in Foundation Models and Their Impact on Healthcare” from researchers at Massachusetts Institute of Technology, Harvard Medical School, University of Washington, Carnegie Mellon University, Seoul National University Hospital, Google, Columbia University, and Johns Hopkins University published on Mar. 3, 2024, states that “non-trivial levels of hallucination persist. These findings underscore the ethical and practical imperative for robust detection and mitigation strategies, establishing a foundation for regulatory policies that prioritize patient safety and maintain clinical integrity as AI becomes more integrated into healthcare. The feedback from clinicians highlights the urgent need for not only technical advances but also for clearer ethical and regulatory guidelines to ensure patient safety.”

When it comes to health care, the risk of hallucinations, incorrect information or out of date source material can be more consequential. For example, Ross (Why the early tests of ChatGPT in medicine miss the mark, Health Tech, Apr. 3, 2023) notes that:
ChatGPT has rocketed into health care like a medical prodigy. The artificial intelligence tool correctly answered more than 80% of board exam questions, showing an impressive depth of knowledge in a field that takes even elite students years to master.
But in the hype-heavy days that followed, experts at Stanford University began to ask the AI questions drawn from real situations in medicine—and got much different results. Almost 60% of its answers either disagreed with human specialists or provided information that wasn't clearly relevant.

When it comes to health care, the risk of hallucinations can be more consequential. For example, a large multi-modal model (LMM) trained on all available data sources could be one where a prompt query asking for treatments of syphilis results in recommendations from long-debunked medieval treatments such as leeches or mercury. While outrageous, this is a realistic scenario if an LMM had been trained on academic manuscripts from the Middle Ages.

If an AI is designed or coerced to return a complete response versus an incomplete one or none at all, a profoundly inaccurate hallucination that is presented as factual (often with fabricated references) can occur and have real-world and very dangerous consequences both to individual safety, trust, and liability.

Subtle instances of misinformation can equally be dangerous and consequential, as they may go unnoticed. One example of a dangerous hallucination that could do real harm and breed mistrust would be AI recommendations based on out-of-date or non-peer-reviewed research. For example, OpenAI's ChatGPT model is based on training data only up until April 2023, so any new or updated research and findings in the medical field would not be reflected in its responses, and there is still little transparency on what data its model is based on and how frequently it's updated. For instance, new research published by the WHO in the Lancet (Anderson et al., Health and cancer risks associated with low levels of alcohol consumption. Lancet Public Health. 2023 January; 8(1): e6-e7) states that there is no safe threshold for alcohol consumption and the risk of cancer and heart disease correlates to alcohol consumption even at low levels. If an end user or health practitioner relied on recommendations from an AI agent based on outdated or training data not certified by medical experts, it could incorrectly suggest that moderate drinking is still safe or ok when the latest scientific evidence recommends otherwise to maximize healthspan and longevity. This could be considered malpractice and present real health risks and expose any companies who misrepresent medical recommendations to liability.

These and other problems are addressed by the technology provided herein. For example, embodiments of the present technology reduce, minimize, and/or eliminate AI/ML shortcomings through use of expert curation systems and methods. The systems and methods employ one or more levels of expert curation to manage information content used in AI system training and, in some embodiments, to audit AI system performance and make changes, as necessary or desired, to maximize performance.

For example, in some embodiments, the systems and methods employ an administrator-controlled, secured, tiered provisioning system to allow only authenticated and verified users (“curators”) to select, upload, ingest, edit, and update content (e.g., websites, papers, articles, tables, charts, audio, media files, transcripts, books, and other digital and analog materials (“sources”)) into a database or knowledge base accessible by a generative AI inference system (e.g., a vector index accessible by a novel RAG (retrieval-augmented-generation)-based AI inference system), an AI language model training database, or the like. In some embodiments, this model is purpose-built only to be based on source data related to predetermined subject matter and/or to only use details from within curated source data and to provide visibility when sources are used outside of the context in which they were curated by “Administrators” to ensure relevance accuracy, and mitigate incorrect responses or hallucinations. “Administrator” and “curators” roles can have a range of customizable permissions, controls, access, and influence on source data and metadata.

The number of tiers and the qualifications of individuals within the tiers will vary depending on the subject matter. In some embodiments, an adjudication board or individual sits at a top level and supervises one or more sub-specialties within the general subject matter area. In some embodiments, the adjudication board or individual nominates, votes for, authenticates, and/or revokes access for tiers that reside below it. In some embodiments, the adjudication board or individual is provided the ability to define, provision, and create discrete databases or knowledge bases (e.g., databases or knowledge bases accessible by a generative AI inference system, curated knowledge bases, language model training databases, etc.) across the sub-specialties that it supervises. In some embodiments, the adjudication board or individual roles are populated by top-tier experts in the field, ideally with organizational management experience.

In some embodiments, residing under the adjudication board is a specialized advisory board or individual that manages a sub-specialty within the general subject matter area managed by the adjudication board. In some embodiments, this tier invites, authenticates, and revokes access for administrators that oversee the recruitment and management of curators for their given field of expertise. In some embodiments, the specialized advisory board or individual is populated by respected and established, and where appropriate, certified, subject matter experts.

In some embodiments, residing under the specialized advisory board is one or more super administrators. In some embodiments, super administrators define, provision, and audit discrete databases or knowledge bases (e.g., databases accessible by a generative AI inference system, curated knowledge bases, language model training databases, etc.) and are authorized to name, invite, edit, and/or revoke administrator roles. In some embodiments, the super administrator has all administrator functionalities. In some embodiments, the super administrator is an experienced subject matter expert with administrative experience, for example, a department dean at a top tier academic institute, or equivalent, in the particular subject matter domain.

In some embodiments, residing under the super administrator is one or more administrators. In some embodiments, administrators audit authorized training databases (e.g., an administrator and associated curators can only access corresponding databases that they have been invited to (e.g., that relate to the subject matter sub-specialty)) and nominate, invite, approve, and revoke super curator roles. In some embodiments, the administrator has all super curator functionalities. In some embodiments, the administrator is an experienced subject matter expert, for example a tenured academic professor, or equivalent, in a subject-specific sub-category.

In some embodiments, residing under the administrator is one or more super curators. In some embodiments, super curators audit, select, ingest, update, and remove training data from authorized training databases. In some embodiments, super curators review all recommendations, ratings, responses, flags, and comments from all user roles. In some embodiments, super curators name, invite, edit, and review curator roles. In some embodiments, super curators have all curator functionalities. In some embodiments, the super curators are subject matter experts, for example, associate or non-tenured professors, or equivalent, in a subject-specific sub-category.

In some embodiments, residing under the super curator is one or more curators. In some embodiments, the curators audit, review, recommend, and rate data sources, prompts, and responses for authorizing training databases. In some embodiments, the curators annotate the source materials. In some embodiments, the curators name, invite, edit, and revoke commenter roles. In some embodiments, the curators have all commentator functionalities. In some embodiments, the curators are subject matter knowledgeable, for example professionals or researchers working in the field of the subject matter sub-category.

In some embodiments, residing under the curator is one or more commentators. In some embodiments, the commentators review, recommend, and rate source data, prompts, and responses for authorized training databases. In some embodiments, the commentators name, invite, edit, and revoke moderator roles. In some embodiments, the commentators have all moderator functionalities. In some embodiments, the commentators are graduate students, or equivalent, in the field of the subject matter sub-category.

In some embodiments, residing under the commentator is one or more moderators. In some embodiments, moderators review end-user prompts and responses, flags, ratings, and recommendations. In some embodiments, moderators name, invite, and revoke end user roles. In some embodiments, moderators are graduate students, or equivalent, in any field related to the subject matter sub-category.

In some embodiments, moderators interact with end users. In some embodiments, end users answer, edit survey questions, and upload personal health data and information. In some embodiments, end users enter text-based prompts and questions and rate and comment on responses and recommendations based on prompts and questions. In some embodiments, end users can enter prompts and questions and rate and comment on responses and recommendations based on prompts and questions using non-text-based means, e.g., audio and video. End users include any user interested in interacting with the system and include professionals, students, researchers, service providers, service users, individuals associated with advocacy groups, government employees, and general individuals.

In some embodiments, failures by the AI system to generate answers, or answers that end-users rate as low-quality, are provided as feedback to the curation system. This feedback refines the model and informs future curation of information to train future iterations of the AI system.

An example of a curation system focused on the field of health care and on the sub-topic of atherosclerosis is provided in Example 1 below to illustrate an implementation of such systems and methods.

The technology finds use in any subject matter area and all of their sub-topic subject matter areas, including, but not limited to, anthropology (e.g., anthropological criminology, anthropological linguistics (e.g., synchronic linguistics, diachronic linguistics, ethnolinguistics, semiotic anthropology, sociolinguistics), anthrozoology, biological anthropology (e.g., gene-culture coevolution, evolutionary anthropology, forensic anthropology, human behavioral ecology, human evolution, medical anthropology, molecular anthropology, neuroanthropology, nutritional anthropology, paleoanthropology, population genetics, primatology) biocultural anthropology, cultural anthropology (e.g., anthropology of development, anthropology of religion, applied anthropology, cognitive anthropology, cyborg anthropology, digital anthropology, digital culture, ecological anthropology, economic anthropology, environmental anthropology, ethnobiology, ethnobotany, ethnography, ethnohistory, ethnology, ethnomuseology, ethnomusicology, feminist anthropology, folklore, kinship, legal anthropology, mythology, missiology, political anthropology, political economic anthropology, psychological anthropology, public anthropology, symbolic anthropology, transpersonal anthropology, urban anthropology) linguistic anthropology, social anthropology (e.g., anthropology of art, anthropology of institutions, anthropology of media, visual anthropology) archaeology (e.g., aerial archaeology, aviation archaeology, anthracology, archaeo-optics, archaeoacoustics, archaeoastronomy, archaeogeography, archaeological culture, archaeological theory (e.g., great ages archaeology, functionalism, processualism, post-processualism, cognitive archaeology, gender archaeology, feminist archaeology) archaeometry (e.g., archaeogenetics, bioarchaeology, computational archaeology, dendrochronology, geoarchaeology, isotope analysis, palynology, radiocarbon dating, zooarchaeology), archaeology of religion and ritual, archaeology of trade, archaeomythology, architectural analytics, battlefield archaeology, calceology, conflict archaeology, data archaeology, digital archaeology, experimental archaeology, environmental archaeology, ethnoarchaeology, forensic archaeology, glyptology, history of archaeology, household archaeology, landscape archaeology and landscape history, manuscriptology, maritime archaeology, media archaeology, modern archaeology (e.g., settlement archaeology) music archaeology, osteology, palaeoarchaeology, paleoanthropology, paleoethnobotany, paleopathology, paleoradiology, taphonomy, urban archaeology, historical archaeology (e.g., prehistoric archaeology, protohistoric archaeology, biblical archaeology, classical archaeology, egyptology, assyriology, etruscology, near eastern archaeology, medieval archaeology, post-medieval archaeology, industrial archaeology, contemporary archaeology), african archaeology, australian archaeology, european archaeology, russian archaeology, archaeology of the americas, archaeology of china, archaeology of israel) history (e.g., african history, (e.g., south african history, egyptian history, nigerian history) pan-american history, north american history (e.g., american history, canadian history, mexican history, cuban history) south american history (e.g., latin american history, brazilian history, colombian history, venezuelan history, peruvian history, argentine history), pre-columbian era (e.g., mayan history, aztec history, inca history, mississippian culture), ancient history (e.g., ancient greek history, ancient roman history (e.g., history of the roman republic, history of the roman empire), ancient egyptian history, ancient chinese history, ancient middle eastern history, asian history (e.g., chinese history, japanese history, korean history, mongolian history, indian history, turkish history, iranian history, philippine history, indonesian history) european history (e.g., british history, french history, german history, dutch history, italian history, spanish history, portuguese history, polish history, balkan history, scandinavian history (e.g., swedish history, norwegian history, danish history, finnish history, icelandic history), russian history) australian history, economic history, environmental history, intellectual history, modern history, political history, scientific history, technological history, world history, public history) linguistics and languages (e.g., classics, languages (e.g., business english, classical language, modern language, standard english, world englishes), applied linguistics, comics studies, composition studies, computational linguistics, discourse analysis, english studies, etymology, grammar, historical linguistics, history of linguistics, interlinguistics, lexicology, linguistic typology, morphology, natural language processing, philosophy of language, philosophy of linguistics, linguistic philosophy, philology, phonetics, phonology, pragmatics, psycholinguistics, semantics, semiotics, sociolinguistics, syntax, terminology science, rhetoric, usage, word usage) philosophy (e.g., meta-philosophy, metaphysics (e.g., ontology, teleology, philosophy of mind (e.g., philosophy of artificial intelligence, philosophy of perception, philosophy of pain), philosophy of space and time, philosophy of action, determinism and free will) epistemology (e.g., justification, reasoning errors), ethics (e.g., meta-ethics, normative ethics (e.g., virtue ethics), applied ethics (e.g., animal rights, bioethics, environmental ethics), moral psychology, descriptive ethics, value theory), aesthetics/philosophy of art, social philosophy and political philosophy (e.g., anarchism, feminist philosophy, libertarianism, marxism), philosophical traditions and schools (e.g., platonism, aristotelianism, analytic philosophy, continental philosophy, eastern philosophy, feminist philosophy), history of philosophy (e.g., ancient philosophy, medieval philosophy (e.g., scholasticism, humanism), modern philosophy, contemporary philosophy), logic (e.g., philosophical logic, mathematical logic), applied philosophy (e.g., philosophy of education, philosophy of history, philosophy of religion, philosophy of language, philosophy of law, philosophy of mathematics, philosophy of music, philosophy of science (e.g., philosophy of social science, philosophy of physics, philosophy of biology, philosophy of chemistry, philosophy of economics, philosophy of psychology), philosophy of engineering, systems philosophy), religion (e.g., abrahamic religions (e.g., christianity (e.g., christian theology), islam/islamic studies, judaism/jewish studies), apologetics, indian religions (e.g., buddhism/buddhist studies, hinduism, jainism, sikhism), east asian religions (e.g., chinese folk religion, confucianism, shinto, daoism, i-kuan tao, caodaism, chondogyo, tenrikyo, oomoto), other religions (e.g., african religions, ancient egyptian religion, native american religions, gnosticism, occult, esotericism, mysticism, spirituality, new religious movements, sumerian religion, zoroastrianism), comparative religion, mythology and folklore, theism, irreligion (e.g., agnosticism, atheism and religious humanism, nontheism), culinary arts (e.g., acquired taste, aftertaste, appetite, artisanal food, cooking, cuisine, culinary arts, culinary tourism, delicacy, diet, flavor, food choice, food pairing, food photography, food preparation, food presentation, food safety, food security, food studies, gastronomy, gourmet, palatability, specialty foods, traditional food) literature (e.g., poetry, comparative literature, english literature, world literature (e.g., american literature, british literature), history of literature (e.g., medieval literature, post-colonial literature, post-modern literature), literary theory (e.g., critical theory, literary criticism, poetics, rhetoric), literary genre, creative writing (e.g., creative nonfiction, fiction writing, non-fiction writing, literary journalism, poetry, screenwriting, playwrighting), performing arts (e.g., music, accompanying, chamber music, church music, musical composition, conducting (e.g., choral conducting, orchestral conducting, wind ensemble conducting), early music, jazz studies, music education, music history, music genre, music theory, musicology (e.g., historical musicology, systematic musicology), ethnomusicology, organology (e.g., organ and historical keyboards, plano, strings, harp, oud, and guitar, singing, woodwinds, brass, and percussion), recording, orchestral studies), dance (e.g., choreography, dance notation, ethnochoreology, history of dance), television (e.g., television studies), theatre (e.g., history, acting, directing, stage design, puppetry, dramaturgy, scenography, musical theatre), film (e.g., animation, live action, filmmaking, film criticism, film genre, film studies, film theory), oral literature (e.g., public speaking, performance poetry, spoken word, storytelling), electronic game (e.g., arcade game, audio game, outline of video games), visual arts (e.g., craft, fine arts, forgery, graphic design, graphic arts (e.g., drawing, painting, photography), sculpture), economics (e.g., agricultural economics, anarchist economics, applied economics, behavioural economics, bioeconomics, business, complexity economics, computational economics, consumer economics, development economics, digital economy, ecological economics, econometrics, economic geography, economic history, economic sector, economic sociology, economic systems, economic value, energy economics, entrepreneurial economics, environmental economics, evolutionary economics, experimental economics, feminist economics, financial economics, financial econometrics, green economics, growth economics, human development theory, industrial organization, information economics, institutional economics, international economics, islamic economics, jel classification codes, knowledge economy, labor economics, law and economics, macroeconomics, managerial economics, market economy, marxian economics, mathematical economics, microeconomics, monetary economics, neuroeconomics, participatory economics, political economy, public finance, public economics, real estate economics, resource economics, social choice theory, socialist economics, socioeconomics, transport economics, welfare economics, geography (e.g., cartography, navigation, human geography (e.g., cultural geography (e.g., feminist geography), economic geography (e.g., development geography), historical geography, time geography, political geography & geopolitics (e.g., military geography, strategic geography), population geography, social geography (e.g., behavioral geography, children's geographies, health geography, tourism geography), urban geography, environmental geography, physical geography (e.g., biogeography, climatology (e.g., palaeoclimatology), coastal geography, geomorphology, geodesy, hydrology/hydrography (e.g., glaciology, limnology, biogeochemistry, oceanography), landscape ecology, palaeogeography), regional geography, remote sensing), ethnic and cultural studies (e.g., cultural studies, ethnic studies, ethnology, culturology, cross-cultural studies), organizational studies (e.g., business economics, business ethics, business studies, decision science, entrepreneurship, human resources management, industrial organization, management, organizational behavior, organization theory, project management, quality control, strategy), political science (e.g., american politics, canadian politics, civics, comparative politics, european studies, geopolitics (political geography), international relations, international organizations, nationalism studies, peace and conflict studies, policy studies, political behavior, political culture, political economy, political history, political philosophy, psephology, public administration (e.g., nonprofit administration, non-governmental organization (ngo) administration), public policy, social choice theory), psychology (e.g., abnormal psychology, applied psychology, asian psychology, biological psychology, black psychology, clinical psychology, clinical neuropsychology, cognitive psychology, community psychology, comparative psychology, conservation psychology, consumer psychology, counseling psychology, criminal psychology, cultural psychology, developmental psychology, differential psychology, ecological psychology, educational psychology, environmental psychology, evolutionary psychology, experimental psychology, group psychology, family psychology, feminine psychology, forensic psychology, forensic developmental psychology, health psychology, humanistic psychology, indigenous psychology, legal psychology, mathematical psychology, media psychology, medical psychology, military psychology, moral psychology and descriptive ethics, music psychology, neuropsychology, occupational psychology, occupational health psychology, organizational psychology, parapsychology (outline), pediatric psychology, pedology (children study), personality psychology, phenomenology, political psychology, positive psychology, problem solving, psychoanalysis, psychobiology, psychometrics, psychology of religion, psychopathology (e.g., child psychopathology), psychophysics, quantitative psychology, rehabilitation psychology, school psychology, social psychology, sport psychology, traffic psychology, transpersonal psychology, travel psychology), sociology (e.g., analytical sociology, applied sociology, political sociology (e.g., public sociology, social engineering, leisure studies), architectural sociology, behavioral sociology, chinese sociology, collective behavior (e.g., activism, social movements), social phenomenon, community informatics (e.g., social network analysis), comparative sociology, conflict theory, critical sociology, cultural sociology, cultural studies, criminology/criminal justice (outline), critical management studies, demography/population, digital sociology, dramaturgical sociology, economic sociology, educational sociology, empirical sociology, environmental sociology, evolutionary sociology, feminist sociology, figurational sociology, futures studies (outline), historical sociology, human ecology, humanistic sociology, industrial sociology, interactionism, internet sociology, interpretive sociology (e.g., phenomenology, ethnomethodology, symbolic interactionism, social constructionism), jealousy sociology, macrosociology, marxist sociology, mathematical sociology, medical sociology, mesosociology, microsociology, military sociology, natural resource sociology, organizational studies, phenomenological sociology, policy sociology, polish sociology, postcolonialism, psychoanalytic sociology, science studies/science and technology studies, sexology, social capital, social change, social conflict theory, social control (e.g., pure sociology), social economy, social philosophy, social psychology, social policy, social research, social transformation (e.g., computational sociology, economic sociology/socioeconomics) (e.g., economic development, social development), sociology of aging, sociology of agriculture, sociology of art, sociology of autism, sociology of childhood, sociology of conflict, sociology of culture, sociology of cyberspace, sociology of deviance, sociology of development, sociology of disaster, sociology of education, sociology of emotions, sociology of fatherhood, sociology of film, sociology of finance, sociology of food, sociology of gender, sociology of generations, sociology of globalization, sociology of government, sociology of health and illness, sociology of human consciousness, sociology of immigration, sociology of knowledge, sociology of language, sociology of law, sociology of leisure, sociology of literature, sociology of markets, sociology of marriage, sociology of motherhood, sociology of music, sociology of natural resources, sociology of organizations, sociology of peace, war, and social conflict, sociology of punishment, sociology of race and ethnic relations, sociology of religion, sociology of risk, sociology of science, sociology of scientific knowledge, sociology of social change, sociology of social movements, sociology of space, sociology of sport, sociology of technology, sociology of terrorism, sociology of the body, sociology of the family, sociology of the history of science, sociology of the internet, sociology of work, social theory, social stratification, sociological theory, sociobiology, sociocybernetics, sociolinguistics, sociomusicology, structural sociology, theoretical sociology, urban studies or urban sociology/rural sociology, victimology, visual sociology), biology (e.g., aerobiology, anatomy (e.g., comparative anatomy, human anatomy), bacteriology, biochemistry, bioinformatics, biophysics, biotechnology, botany (e.g., ethnobotany, phycology), cell biology, chronobiology, cognitive biology, computational biology, conservation biology, cryobiology, developmental biology (e.g., embryology, gerontology, teratology), ecology (e.g., agroecology, ethnoecology, human ecology, landscape ecology), forensic biology, genetics (e.g., behavioral genetics, molecular genetics, population genetics), geobiology, endocrinology, evolution (e.g., systematics, taxonomy), histology, human biology, immunology, limnology, linnaean taxonomy, marine biology, mathematical biology, microbiology, molecular biology, mycology, neuroscience (e.g., behavioral neuroscience, neurophysics, computational neuroscience), nutrition, paleobiology (e.g., paleontology), parasitology, pathology (e.g., anatomical pathology, clinical pathology, dermatopathology, forensic pathology, hematopathology, histopathology, molecular pathology, surgical pathology, phytopathology), physiology (e.g., human physiology (e.g., exercise physiology), population biology, psychobiology, quantum biology, sociobiology, structural biology, systems biology, theoretical biology, toxicology, virology (e.g., molecular virology), xenobiology, zoology (e.g., animal communications, apiology, arachnology, arthropodology, batrachology, bryozoology, carcinology, cetology, cnidariology, entomology (e.g., forensic entomology), ethnozoology, ethology, helminthology, herpetology, ichthyology (outline), invertebrate zoology, mammalogy (e.g., cynology, felinology), malacology (e.g., conchology, limacology, teuthology), myriapodology, myrmecology, nematology, neuroethology, oology, ornithology (outline), planktology, primatology, zootomy, zoosemiotics), chemistry (e.g., agrochemistry, analytical chemistry, astrochemistry, atmospheric chemistry, biochemistry (outline), catalysts, chemical engineering (outline), chemical biology, chemical physics, cheminformatics, computational chemistry, cosmochemistry, environmental chemistry, femtochemistry, flavor, flow chemistry, forensic chemistry, geochemistry, green chemistry, histochemistry, hydrogenation, immunochemistry, inorganic chemistry, marine chemistry, mathematical chemistry, mechanochemistry, medicinal chemistry, molecular biology, molecular mechanics, nanotechnology, natural product chemistry, neurochemistry, nuclear chemistry, oenology, organic chemistry (outline), organometallic chemistry, petrochemistry, pharmacology, photochemistry, physical chemistry (e.g., electrochemistry, physical organic chemistry), phytochemistry, polymer chemistry, quantum chemistry, radiochemistry, soil chemistry, solid-state chemistry, sonochemistry, supramolecular chemistry, surface chemistry, synthetic chemistry, systems chemistry, theoretical chemistry, thermochemistry), earth sciences (e.g., atmospheric science, climatology, ecology, edaphology, environmental science, environmental chemistry, forensic geology, gemology, geobiology, geodesy, geography, geology, geochemistry, geomorphology, geophysics, glaciology, hydrogeology, hydrology, meteorology, mineralogy, limnology, oceanography, pedology, paleontology (e.g., paleobiology, paleoecology), petrology, planetary science, sedimentology, seismology, soil science, speleology, tectonics, volcanology), physics (e.g., acoustics (e.g., quantum acoustics), agrophysics, applied physics (e.g., accelerator physics, communication physics), astrophysics, atmospheric physics (e.g., atmospheric electricity), atomic, molecular, and optical physics, atomic physics, biophysics (outline) (e.g., neurophysics), chemical physics, classical physics, computational physics, condensed matter physics, cryogenics, digital physics, dynamics (e.g., analytical dynamics, astrodynamics, brownian dynamics, file dynamics, flight dynamics, fluid dynamics (e.g., aerodynamics, hydrodynamics), fractional dynamics, geodynamics, molecular dynamics, newtonian dynamics, langevin dynamics, quantum chromodynamics, quantum electrodynamics, relativistic dynamics, stellar dynamics, system dynamics, thermodynamics, vehicle dynamics), econophysics, electromagnetism (e.g., electricity (e.g., electrostatic), magnetism), engineering physics, experimental physics, geophysics (e.g., biogeophysics, geomagnetism), kinematics (e.g., fluid kinematics, relativistic kinematics), kinetics (e.g., electrokinetics, homeokinetics), laser physics, materials physics, mathematical physics, medical physics, mechanics (e.g., analytical mechanics, applied mechanics, ballistics, biomechanics, celestial mechanics, classical mechanics, continuum mechanics, fluid mechanics (e.g., compressible flow, gas mechanics), fracture mechanics, hamiltonian mechanics, hydraulics, lagrangian mechanics, matrix mechanics, molecular mechanics, optomechanics, particle mechanics, quantum mechanics, relativistic mechanics, relativistic quantum mechanics, soil mechanics, solid mechanics, statistical mechanics (e.g., quantum statistical mechanics), mineral physics, molecular physics, nuclear physics, optics (e.g., geometrical optics, physical optics, quantum optics), particle physics, petrophysics, photonics, physical chemistry, plasma physics, polymer physics, quantum physics (e.g., quantum technology), radiophysics, relativity (e.g., general relativity, special relativity), social physics, soil physics, solid state physics, spintronics, statics (e.g., fluid statics), statistical physics, surface physics, theoretical physics (e.g., quantum field theory, quantum gravity), thermal physics), space sciences (e.g., aerospace engineering (e.g., aerospace architecture, aerospace physiology, aerospace manufacturing, astronautics (e.g., space architecture, space colonization, space commercialization (e.g., space-based economy, space industry, space manufacturing, space tourism), space environment, space logistics, space food, space medicine (e.g., neuroscience in space), space religion, space sex, space survival, space warfare, space writing), aeronautics, control engineering, human spaceflight, robotic spacecraft, space corrosion), astronomy (e.g., archaeoastronomy, astrometry, amateur astronomy, forensic astronomy, extragalactic astronomy, galactic astronomy, high-energy astronomy, observational astronomy (e.g., radio astronomy, microwave astronomy, submillimetre astronomy, infrared astronomy, optical astronomy, uv astronomy, x-ray astronomy, gamma-ray astronomy, cosmic-ray astronomy, neutrino astronomy, gravitational wave astronomy), photometry, spectroscopy, stellar astronomy (e.g., solar astronomy), space technology (e.g., space telescopes, space-based radar, space-based solar power, spacecraft design, spacecraft propulsion), asteroid-impact avoidance, astrobiology, astrobotany, astrochemistry (e.g., theoretical astronomy), cosmochemistry, cosmology (e.g., physical cosmology), micro-g environment research, remote sensing, space archaeology, space exploration, space law, space nuclear power), astrophysics (e.g., celestial mechanics, compact objects, computational astrophysics, gravitational astronomy (e.g., black holes), interstellar medium, numerical simulations (e.g., astrophysical plasma, galaxy formation and evolution, high-energy astrophysics, hydrodynamics, magnetohydrodynamics, star formation), orbital mechanics, physical cosmology, relativistic astrophysics, stellar astrophysics (e.g., helioseismology, solar physics, stellar evolution, stellar nucleosynthesis), space plasma physics), planetary science (e.g., atmospheric science, exoplanetology, planetary formation, planetary rings, magnetospheres, planetary geology, planetary surfaces, small solar system bodies), computer sciences (e.g., theory of computation (e.g., automata theory (formal languages), computability theory, computational complexity theory, concurrency theory), vlsi design, operating systems, algorithms (e.g., randomized algorithms, distributed algorithms, parallel algorithms, computational geometry), database, data science, data structures, computer architecture, computer communications (networks) (e.g., information theory, internet, world wide web, wireless computing (mobile computing), ubiquitous computing, cloud computing), computer program, computer programming, computer security and reliability (e.g., cryptanalysis, cryptography, fault-tolerant computing), distributed computing (e.g., grid computing), parallel computing (e.g., high-performance computing), quantum computing, computer graphics (e.g., image processing, scientific visualization), software engineering (e.g., formal methods (formal verification)), programming languages (e.g., programming paradigms (e.g., imperative programming, object-oriented programming, functional programming, logic programming, concurrent programming), program semantics, type theory, compilers), human-computer interaction, information science (e.g., data management, data mining, database (e.g., relational database, distributed database, object database), information retrieval, information management, information system, information technology, knowledge management, multimedia, hypermedia (e.g., sound and music computing), quantum information), theoretical computer science, artificial intelligence (e.g., cognitive science (e.g., automated reasoning, machine learning (e.g., artificial neural network, support vector machine), natural language processing (computational linguistics), computer vision), expert systems, robotics), computing in mathematics, natural sciences, engineering, and medicine (e.g., numerical analysis, algebraic (symbolic) computation, computational number theory, computational mathematics, scientific computing (computational science), computational biology (bioinformatics), computational physics, computational chemistry, computational neuroscience, computer-aided engineering (e.g., finite element analysis, computational fluid dynamics), computing in social sciences, arts, humanities, and professions (e.g., computational economics, computational sociology, computational finance, digital humanities (humanities computing), history of computer hardware, history of computer science (outline), humanistic informatics, community informatics), logic (e.g., mathematical logic (e.g., set theory, proof theory, model theory, recursion theory, modal logic, intuitionistic logic), philosophical logic (e.g., logical reasoning, modal logic (e.g., deontic logic, doxastic logic), logic in computer science (e.g., programming language semantics, formal methods (formal verification), type theory, logic programming, multi-valued logic (e.g., fuzzy logic), pure mathematics (e.g., algebra (e.g., group theory, ring theory (e.g., commutative algebra), field theory, linear algebra (vector space), multilinear algebra, universal algebra, homological algebra, differential algebra, lattice theory (order theory), representation theory, k-theory, category theory (e.g., topos theory), analysis (e.g., real analysis (e.g., calculus), complex analysis, functional analysis (e.g., operator theory), non-standard analysis, harmonic analysis (e.g., fourier analysis), p-adic analysis, ordinary differential equations, partial differential equations), probability theory (e.g., measure theory (e.g., integral geometry), ergodic theory, stochastic process), geometry and topology (e.g., general topology, algebraic topology, geometric topology, differential topology, algebraic geometry, projective geometry, affine geometry, non-euclidean geometry, convex geometry, discrete geometry, integral geometry, euclidean geometry, finite geometry, galois geometry, noncommutative geometry, solid geometry, trigonometry), number theory (e.g., analytic number theory, algebraic number theory, geometric number theory, arithmetic, arithmetic combinatorics), logic and foundations of mathematics (e.g., set theory, proof theory, model theory, recursion theory, modal logic, intuitionistic logic), applied mathematics (e.g., approximation theory, computational mathematics, numerical analysis, operations research (e.g., mathematical optimization, linear programming, dynamic programming, assignment problem, decision analysis, inventory theory, scheduling, real options analysis, systems analysis, stochastic processes, optimal maintenance), dynamical systems (e.g., chaos theory, fractal geometry), mathematical physics (e.g., quantum mechanics, quantum field theory, quantum gravity (e.g., string theory), statistical mechanics), theory of computation (e.g., computational complexity theory), information theory, cryptography, steganography, combinatorics (e.g., coding theory), graph theory, game theory), statistics (e.g., mathematical statistics, econometrics, actuarial science, demography, computational statistics (e.g., data mining, regression, simulation (e.g., bootstrap (statistics)), design of experiments (e.g., block design and analysis of variance, response surface methodology), sample survey (e.g., sampling theory), statistical modelling (e.g., biostatistics (e.g., epidemiology), multivariate analysis (e.g., structural equation model, time series), reliability theory, quality control), statistical theory (e.g., decision theory, mathematical statistics (e.g., probability), survey methodology), systems science (e.g., network science, chaos theory, conceptual systems, communications system, complex system, cybernetics (e.g., biocybernetics, engineering cybernetics, management cybernetics, medical cybernetics, new cybernetics, second-order cybernetics, sociocybernetics), control theory (e.g., affect control theory, control engineering, control systems, dynamical systems, perceptual control theory), operations research, systems biology (e.g., computational systems biology, synthetic biology, systems immunology, systems neuroscience), systems chemistry, system dynamics (e.g., social dynamics), systems ecology (e.g., ecosystem ecology), systems engineering (e.g., biological systems engineering, earth systems engineering and management, enterprise systems engineering, systems analysis), systems theory in anthropology, systems psychology (e.g., ergonomics, family systems theory, systemic therapy), systems theory (e.g., biochemical systems theory, ecological systems theory, developmental systems theory, general systems theory, living systems theory, lti system theory, sociotechnical systems theory, mathematical system theory, world-systems theory), agriculture (e.g., aeroponics, agroecology, agrology, agronomy, animal husbandry (animal science) (e.g., beekeeping (apiculture)), anthroponics, agricultural economics, agricultural engineering (e.g., biological systems engineering, food engineering), aquaculture, aquaponics, enology, entomology, fogponics, food science (e.g., culinary arts), forestry, horticulture, hydrology, hydroponics, pedology, plant science (e.g., pomology), pest control, purification, viticulture), architecture and design (e.g., architecture (e.g., interior architecture, landscape architecture, architectural analytics), historic preservation, interior design (interior architecture), landscape architecture (landscape planning), landscape design, urban planning (urban design), visual communication (e.g., graphic design (e.g., type design), technical drawing), industrial design (product design) (e.g., ergonomics, toy and amusement design), user experience design (e.g., interaction design, information architecture, user interface design, user experience evaluation), decorative arts, fashion design, textile design), business (e.g., accounting (e.g., accounting research, accounting scholarship), business administration, business analysis, business ethics, business law, e-business, entrepreneurship, finance, industrial and labor relations (e.g., collective bargaining, human resources, organizational studies, labor economics, labor history), information systems (business informatics) (e.g., management information systems, health informatics), information technology, international trade, management, marketing, operations management, purchasing, risk management and insurance, systems science), divinity (e.g., canon law, church history, field ministry (e.g., pastoral counseling, pastoral theology, religious education techniques, homiletics, liturgy, sacred music, missiology), hermeneutics, scriptural study and languages (e.g., biblical hebrew, biblical studies/sacred scripture, vedic study, new testament greek, latin, old church slavonic), theology (e.g., dogmatic theology, ecclesiology, sacramental theology, systematic theology, christian ethics, hindu ethics, moral theology, historical theology), education (e.g., comparative education, critical pedagogy, curriculum and instruction (e.g., alternative education, early childhood education, elementary education, secondary education, higher education, mastery learning, cooperative learning, agricultural education, art education, bilingual education, chemistry education, counselor education, language education, legal education, mathematics education, medical education, military education and training, music education, nursing education, outdoor education, peace education, physical education/sports coaching, physics education, reading education, religious education, science education, special education, sex education, sociology of education, technology education, vocational education, educational leadership, educational philosophy, educational psychology, educational technology, distance education), chemical engineering (e.g., biocatalysts, bioengineering (e.g., biochemical engineering, biomolecular engineering, bionics), catalysis, materials engineering, molecular engineering, nanotechnology, polymer engineering, process design (e.g., petroleum engineering, nuclear engineering, food engineering), process engineering, reaction engineering, thermodynamics, transport phenomena), civil engineering (e.g., agricultural engineering, coastal engineering, construction, earthquake engineering, ecological engineering, environmental engineering, geotechnical engineering (e.g., engineering geology), hydraulic engineering, infrastructure, mining engineering, transportation engineering (e.g., highway engineering), structural engineering (e.g., architectural engineering), structural mechanics, surveying), electrical engineering (e.g., applied physics, computer engineering, computer science, control systems engineering (e.g., control theory), electronic engineering (e.g., electronics, instrumentation engineering), engineering physics (e.g., photonics), information theory, mechatronics, power engineering, robotics (e.g., microbotics), semiconductors, telecommunications engineering, quantum computing), materials science and engineering (e.g., biomaterials, ceramic engineering, corrosion engineering, crystallography, nanomaterials, photonics, physical metallurgy, polymer engineering, polymer science, semiconductors) mechanical engineering (e.g., aerospace engineering (e.g., aeronautics, astronautics), acoustical engineering, automotive engineering, biomedical engineering (e.g., biomechanical engineering), continuum mechanics, fluid mechanics, heat transfer, industrial engineering, manufacturing engineering, marine engineering, mass transfer, mechatronics, nanoengineering, ocean engineering, optical engineering, robotics, thermal engineering, thermodynamics), environmental studies and forestry (e.g., environmental management (e.g., coastal management, fisheries management, land management, natural resource management, waste management, wildlife management, environmental policy, wildlife observation, recreation ecology, silviculture, sustainability studies (e.g., sustainable development), toxicology, ecology), family and consumer science (e.g., consumer education, housing, interior design, nutrition (e.g., foodservice management), textiles), human physical performance and recreation (e.g., biomechanics/sports biomechanics, sports coaching, escapology, ergonomics, physical fitness (e.g., aerobics, personal trainer/personal fitness training), game design, exercise physiology, kinesiology/exercise physiology/performance science, leisure studies, navigation, outdoor activity, physical activity, physical education/pedagogy, sociology of sport, sexology, sports/exercise, sports journalism/sportscasting, sport management (e.g., athletic director), sport psychology, sports medicine (e.g., athletic training), survival skills (e.g., batoning, bushcraft, scoutcraft, woodcraft), toy and amusement design), journalism, media studies and communication (e.g., journalism (e.g., broadcast journalism, digital journalism, literary journalism, new media journalism, print journalism, sports journalism/sportscasting), media studies (mass media) (e.g., newspaper, magazine, radio, television (e.g., television studies), film (e.g., film studies), game studies, fan studies), narratology (e.g., internet), communication studies (e.g., advertising, animal communication, communication design, conspiracy theory, digital media, electronic media, environmental communication, hoax, information theory, intercultural communication, marketing (outline), mass communication, nonverbal communication, organizational communication, popular culture studies, propaganda, public relations (outline), speech communication, technical writing, translation), law (e.g., legal management (academic discipline) (e.g., corporate law, mercantile law, business law), administrative law, canon law, comparative law, constitutional law, competition law, criminal law (e.g., criminal procedure, criminal justice (e.g., police science, forensic science), islamic law, jewish law, jurisprudence (philosophy of law), civil law (e.g., admiralty law, animal law/animal rights, common law, corporations, civil procedure, contract law, environmental law, family law, federal law, international law (e.g., public international law, supranational law), labor law, paralegal studies, property law, tax law, tort law), law enforcement, procedural law, substantive law), library and museum studies (e.g., archival science, archivist, bibliographic databases, bibliometrics, bookmobile, cataloging (e.g., citation analysis), categorization, classification (e.g., library classification, taxonomic classification, scientific classification, statistical classification, security classification, film classification), collections care, collection management, collection management policy, conservation science, conservation and restoration of cultural heritage, curator, data storage, database management, data modeling, digital preservation, dissemination, film preservation, five laws of library science, historic preservation, history of library science, human-computer interaction, indexer, informatics, information architecture, information broker, information literacy, information retrieval, information science (outline), information systems and technology, integrated library system, interlibrary loan, knowledge engineering, knowledge management, library, library binding, library circulation, library instruction, library portal, library technical services, management, mass deacidification, museology, museum education, museum administration, object conservation, preservation, prospect research, readers' advisory, records management, reference, reference desk, reference management software, registrar, research methods, slow fire, special library, statistics), medicine and health (e.g., alternative medicine, anesthesiology, cleaning, clinical laboratory sciences/clinical pathology/laboratory medicine (e.g., clinical biochemistry, cytogenetics, cytohematology, cytology, haemostasiology, histology, clinical immunology, clinical microbiology, molecular genetics, parasitology), clinical physiology, cosmetology, decontamination, dentistry (e.g., dental hygiene and epidemiology, dental surgery, endodontics, orthodontics, oral and maxillofacial surgery, periodontics, prosthodontics, implantology), dermatology, emergency medicine, health informatics/clinical informatics, music therapy, nursing, nutrition (outline) and dietetics, optometry, orthoptics, osteopathy, physiotherapy, occupational therapy, speech and language pathology, internal medicine (e.g., preventive medicine, cardiology (e.g., cardiac electrophysiology), dermatology, pulmonology (e.g., medical toxicology), endocrinology, gastroenterology (e.g., hepatology), oncology, geriatrics, gynaecology, hematology, infectious disease, nephrology, neurology, neurosurgery, obstetrics, ophthalmology (e.g., neuro-ophthalmology), orthopedic surgery, otolaryngology, pathology, pediatrics), pharmacy, pharmaceutical sciences, pharmacognosy, physical fitness (e.g., aerobics, personal fitness training, kinesiology/exercise physiology/performance science), physical therapy, podiatry, primary care (e.g., general practice), psychiatry, psychology, psychosomatic, psychotherapy, public health, radiology, recreation therapy, rehabilitation medicine, respiratory medicine (e.g., pulmonology, sleep medicine), respiratory therapy, rheumatology, sports medicine, sterilization (microbiology), surgery (e.g., bariatric surgery, cardiothoracic surgery, neurosurgery, plastic surgery, trauma surgery, traumatology), traditional medicine, therapy, urology (e.g., andrology), veterinary medicine), military sciences (e.g., amphibious warfare, artillery, battlespace (e.g., air, information, land, sea, space), campaigning, military engineering, doctrine, espionage, game theory, grand strategy (e.g., containment, limited war, military science, philosophy of war, strategic studies, total war, war), leadership, logistics (e.g., materiel, supply chain management), military operation, military history (e.g., prehistoric, ancient, medieval, early modern, industrial, modern, fourth-generation warfare), military intelligence, military law, military medicine, naval science (e.g., naval engineering, naval tactics, naval architecture), organization (e.g., command and control, doctrine, education and training, engineers, intelligence, ranks, staff, technology and equipment, military exercises, military simulation, military sports), strategy (e.g., attrition, deception, defensive, offensive, counter-offensive, maneuver, goal, naval), tactics (e.g., aerial, battle, cavalry, charge, counter-attack, counter-insurgency, counter-intelligence, counter-terrorism, foxhole, endemic warfare, guerrilla warfare, infiltration, irregular warfare, morale, naval tactics, siege, surgical strike, tactical objective, trench warfare), military weapons (e.g., armor, artillery, biological, cavalry, conventional, chemical, cyber, economic, electronic, infantry, nuclear, psychological, unconventional), other military (e.g., arms control, arms race, assassination, asymmetric warfare, civil defense, clandestine operation, collateral damage, cold war (general term), combat, covert operation, cyberwarfare, defense industry, disarmament, intelligence agency, laws of war, mercenary, military campaign, military operation, mock combat, network-centric warfare, paramilitary, principles of war, private defense agency, private military company, proxy war, religious war, security, special forces, special operations, theater (warfare), theft, undercover, war crimes, warrior), public administration (e.g., civil service, corrections, conservation biology, criminal justice (outline), disaster research, disaster response, emergency management, emergency services, fire safety (structural fire protection), fire ecology (wildland fire management), governmental affairs, international affairs, law enforcement, peace and conflict studies, police science, policy studies (e.g., policy analysis), public administration (e.g., nonprofit administration, non-governmental organization (ngo) administration, public policy doctrine, public policy school, regulation), public safety, public service), public policy (e.g., agricultural policy, commercial policy, cultural policy, domestic policy, drug policy (e.g., drug policy reform), economic policy (e.g., fiscal policy, incomes policy, industrial policy, investment policy, monetary policy, tax policy), education policy, energy policy (e.g., nuclear energy policy, renewable energy policy), environmental policy, food policy, foreign policy, governance, health policy (e.g., pharmaceutical policy, vaccination policy), housing policy, immigration policy, knowledge policy, language policy, military policy, science policy (e.g., climate change policy, stem cell research policy, space policy, technology policy), security policy, social policy, public policy by country), social work (e.g., child welfare, community practice (e.g., community organizing, social policy), human services, corrections, gerontology, medical social work, mental health, school social work), transportation (e.g., highway safety, infographics, intermodal transportation studies, logistics, marine transportation (e.g., port management, seafaring), operations research, mass transit, travel, vehicles).

The systems and methods may employ any one or more of the curation tiers identified above. In some embodiments, responsibilities for the above tiers are consolidated into a single tier. For example, a single administrator tier may take on the activities and responsibilities of the above listed super administrator and administrator tiers. Likewise, a single curator tier may take on the activities and responsibilities of the super curator and curator tiers.

In some embodiments, the system implements a topic-based architecture for organizing source materials, wherein each topic corresponds to a primary curator's domain of expertise. This architectural approach permits a distributed curation model in which a primary curator can programmatically delegate access privileges to additional curators for collaborative contribution within a specific topic domain. In some embodiments, the query processing subsystem employs multiple algorithmic techniques to match incoming queries to the most relevant topic or topics, including: qualification-based matching that evaluates curator expertise profiles against query content; vector-based semantic similarity calculations between query embeddings and topic source material embeddings; structured tag and keyword association systems; and other computational matching methodologies.

In some embodiments, within each topic, the system maintains a document collection architecture wherein curator-assembled source materials undergo a multi-layer validation process. The validation processing system captures both document-level annotations and granular annotations at the subsection, paragraph, and sentence levels. The annotation system implements a rating schema that programmatically determines content inclusion or exclusion parameters when generating responses related to the topic. In some embodiments, the system also processes natural language annotations, which are computationally incorporated as contextual instructions or supplementary information during response generation. In some embodiments, the annotation subsystem is configured to permit real-time updates, with changes propagating through the system's vector indices and retrieval mechanisms. In some embodiments, the system includes a collaboration module that collates annotation contributions for systematic review by the primary curator.

In some embodiments, the system additionally implements automated annotation generation capabilities. The automated annotation subsystem employs computational logic to identify and exclude document sections with low information value for training or response generation, such as pagination elements, standard disclaimers, or funding acknowledgments that do not contribute substantive content.

In some embodiments, the system incorporates a content coverage analysis module wherein curators define a structured outline of required content components for a given topic. For example, a topic focused on “increasing healthspan via improved musculoskeletal health” might specify a computational outline including parameters for strength training methodologies, cardiovascular exercise protocols, mobility enhancement techniques, and related subcategories. This structured outline permits the system to algorithmically analyze the coverage provided by source documents, accounting for inclusion/exclusion parameters derived from annotations. When document exclusions result in coverage gaps, the system generates automated alerts to curators identifying specific content domains requiring additional source materials.

Information may be processed and managed by the systems and methods in any manner that achieves the desired outcomes. In some embodiments, administrators or curators identify and nominate curated material (e.g., training data material (e.g., a newly published peer research paper in a particular journal on a specific topic)). Sources of discovery can come from individual news intake, professional networks, or “bubbled up” from end user, moderator, or commentor recommendations.

Once a “quorum” of endorsements on nominated source material by a “committee” (e.g., a predefined number or percent) of curators or administrators in a given specialization field is reached (e.g., based on an internal “Peer Reviewed” protocol), source material is sent to a “Data Quality Control Team” (Data QC Team) or an automated QC system to ingest into a training database. In some embodiments, the Data QC Team or automated QC system transforms source material into standardized, tokenized, machine-readable format with all associated metadata (see below), including detailed source material attribution. Once source material is ingested, curators/administrators who nominated and voted in favor are notified to prompt LLM to test and verify that source material is correctly referenced in responses. The Data QC team or automated QC system verifies that all appropriate metadata attributions and citations are correctly tracked and displayed and that the changelog reflecting these updates references all modifications, contributions, and attributions. The curators and the Data QC team or automated QC system provides expert validation, compares views to identify contradictions between sources, tracks for evolving guidelines, and provides a mechanism for gauging and ensuring confidence in the source materials.

Source materials can originate from multiple inputs and vectors. The source materials are not limited by media type or source. Source material may be from websites, papers, media files, transcripts, books, articles, presentations, and other digital and analog materials and may include tables, charts, audio, videos, images, and the like. Exemplary source materials include, but are not limited to, journal publications, surgery or lecture videos, conference presentations including text, audio, video, or images therefrom, podcasts, videos or other multimedia accompanying educational materials or courses, diagnostic images, medical calculators or decision aids, virtual models, exercise videos based on individual health conditions, medical pamphlets, recorded consultations with experts, and the like.

Traditional “top-down” discovery may be via journal publications and professional networks through which curators find and nominate peer-reviewed studies or other verified source materials directly. As the number of end users grows to thousands and millions of users, bottom-up or user-generated discovery and selection of source material occurs through the analysis of user interactions, prompts, responses, ratings, and comments at scale; enabled by AI but with humans in the loop at every phase.

Source materials can be selected using a variety of different overarching approaches. In some embodiments, the source materials are selected based on an evidence-based hierarchical approach. Such an approach, for example, prioritizes peer-reviewed source materials, includes guidelines from major organizations, incorporates educational materials and reference works, and selectively adds high-quality websites in conjunction with systematic reviews and meta-analyses. Additionally or alternatively, diversity considerations may be used when selecting source materials. These considerations ensure representation across different specialties, include information relevant to diverse populations (e.g., age, gender, ethnicity), balance technical content with user-friendly materials, and consider regional variations, when appropriate.

Source materials can be annotated during, after, or concurrently with discovery and selection. For example, the source materials can be annotated to for a particular specialty category (e.g., cardiology, pediatrics, etc.), evidence level classification (e.g., randomized controlled trials, observational study, expert consensus), publication date and currency indicators, target audience (e.g., clinicians vs. patients), geographical relevance, confidence/certainty level of medical claims.

In some embodiments, the quality control system comprises a document processing pipeline optimized for handling documents such as PDFs, common in scientific literature and medical/health research journals. In some embodiments, each page of a PDF is first converted to an image using conversion tools. Concurrently, text and non-text components (e.g., images, charts, graphs) are extracted. In some embodiments, the system analyzes the page layout using tools such as AWS Textract, which identifies how text flows across page layout elements such as columns, with possible components such as tables, charts, and captions interrupting the flow of text, and extracts text and components into blocks. For blocks which are not text components (e.g., charts, images), the system may convert to text by using an LLM to describe the component. Optionally, the text surrounding this component may be provided as context in generating a description of the chart, image, etc. In some embodiments, the system extracts text from ordered blocks and combines into a single, accurately flowing body of text, organized by pages of the original document. The page number of the original document is stored as page-level metadata.

In some embodiments, the quality control system converts page data into document chunks for reference by the AI system. Several chunk strategies may be used in parallel, to optimize access to the document text for a variety of purposes. In one example strategy, the system splits document text into “chunks” based on semantic similarity, using tools such as llama-index. This results in chunks based on a similar/cohesive meaning of text within each chunk. This approach ensures that the document text is split into pieces, each of which contain adequate context around information within that text. In another example strategy, the system splits document text by sentences or paragraphs, resulting in a more consistent sizing of chunks, which is important for balancing tradeoffs during retrieval. In some embodiments, each chunk (including chunks from multiple chunking strategies) is converted into a vector representation and stored in a vector index such as OpenSearch, representing a standard approach for retrieval augmented generation (RAG).

An example of top-down material that would pass peer review: Paradigm shifts are occasionally discovered (e.g., prions are responsible for bovine spongiform encephalopathy (BSE)). Such shifts may be met with initial skepticism but are eventually accepted based on the weight of data. The source material, absent curation, will include historical inaccurate information and skepticism. Expert curation permits selection of training data or editing of training data consistent with current knowledge and understanding of the topic in question.

An example of top-down material that would not pass peer review: an on market, regulatory approved drug is found to be dangerous once used in the general population and is pulled off the market. An AI-system may still recommend the drug based on the historical record, which may include peer reviewed publications suggesting that it is safe. The curation employed in the present technology provides a system that would not make such a recommendation.

An example of bottom-up material that would pass peer review: ayurvedic medicine for centuries recommended an herb with a snake-like root called Rauvolfia serpentina to reduce blood pressure. It was found that an extract from Rauvolfia serpentina is a safe and effective treatment for hypertension. The plant was used by many physicians throughout India in the 1940s and then was used throughout the world in the 1950s, including in the United States and Canada. If a critical mass of end-users recommended Rauvolfia serpentina, and it “bubbled up” to moderators and curators, after verifying its medical efficacy and passing internal “peer review” as described above, it would be selected to be ingested as training data source material.

An example of bottom-up material that would not pass peer review: an end-user recommends using the herb “Mau Huang” for lowering blood pressure (BP) rather than the FDA approved drug Losartan Potassium because the patient is experiencing side effects. If a critical mass of end-users recommended “Mau Huang” and it “bubbled up” to moderators and curators, they would conduct an internal “peer review” and the medical efficacy of “Mau Huang” would not be verified. The curators would find that the herb contains ephedra and raises blood pressure. The combination of quitting the known BP-lowering drug and taking the Chinese herb can cause extremely high BP and strokes. If there was no filtering and moderating of user recommendations, or the training data somehow got “contaminated” or “confused” by a surge of potentially harmful or unproven recommendations and did not take into account other medications or preexisting conditions (or at least raise these factors and risks to the end user) it could pose real harm. The expertise provided by the curation system ensures that the training data would not lead to such a recommendation.

In some embodiments, a participant in the curation system would be responsible for inputting all databases and training the system how to use, analyze, and interpret the data in the database. Examples in health include the Framingham study, HANES, Odyssey, databases, the Harvard nutrition study database, or the Norwegian Mother/Child database. Participants in the curation system would be responsible for choosing which databases would be input and which excluded in addition to which technologies or professional skills would be needed to be input into the system to train it in interpreting the database. Such as, as exemplified in the above case, epidemiology, biostatistics, operational research, genomics, longevity science, actuarial science, etc.

Widely implemented, the technology benefits from numerous participants in the curation system. An effective system does not require that individual participants in the curation system dedicate full-time efforts. Traditional employment models are not optimal for staffing such a curation system. In some embodiments, provided herein are systems and methods for incentivizing individuals and groups to participate in the curation systems and methods and to provide high quality contributions to the curation systems and methods. In some embodiments, the incentivization comprises financial rewards (e.g., via revenue sharing, direct financial benefits, indirect financial benefits) and/or reputational rewards (e.g., via ranking, scoring, or other reputational indices that provide status, prestige, or recognition).

For example, in some embodiments, provided herein are systems and methods for revenue sharing based on an evaluation of participant contributions, work quality, and content quality. The systems and methods may evaluate and provide revenue sharing for any and all participants in the expert curation system, including end-users as well as any other person or entity that interacts with the systems or methods or generates or provide content evaluated by the systems and methods (e.g., in some embodiments, is applied to sources (e.g. publishers, researchers, universities, intellectual property (IP) owners, etc.)

In some embodiments, the revenue sharing systems and methods employ a “share of voice” assessment to weigh contributions and work quality. For example, in some embodiments, various inputs (“sources” and “curators” surrounding metadata) are assessed to derive responses to user prompts and to calculate the values of data input, based on one or more of the proportion of underlying citations and attributions, the aggregate metadata of user interaction with the AI (including proportionally weighted rating of response from “curators” and “consumers”), to determine the distribution of proportional revenues to “curators” and “sources” derived from any “customer” activity under a range of revenue models, including paid subscriptions, software as a service, affiliate sales, and 3rd party software licensing.

In some embodiments, data usage metadata is used to calculate revenue sharing distributions.

In some embodiments, each piece of data is placed in a retrieval augmented generation output module modified to increase a counter every time the retrieval algorithm considers the data. This is an inline tally sheet of use within the model. The counter can be retrieved at any time to compute appropriate payout.

In some embodiments, a token is issued for every data input, and this token stores all interactions with it on a ledger. The size of the ledger in bits is then the value of the token. The aggregate size of all tokens associated with the curator is the curator's value.

In some embodiments, the revenue sharing assessment includes selection of one or more separate denominators, representing, for example, profit, EBITDA, or top-line revenue, associated with a group of curators. In some embodiments, a single denominator, representing total revenue from all sources, is selected for use in assessing revenue sharing for all participants in the curation systems and methods. In some embodiments, multiple different denominators are selected. For example, separate denominators may be selected based on different revenue sources or based on different subject matter, geographic areas, or any other desired demarcations.

In some embodiments, different curation system participant tiers or individuals (e.g., advisory board members, administrators, curators, moderators, etc.) are assigned a royalty rate appropriate for their level of expertise and responsibility. In some embodiments, the royalty rate is independent of the amount of or quality of work. For example, a super administrator and curator that otherwise have identical contribution scores may receive a different percentage of the revenue based on the royalty rate assigned to them or their position.

In some embodiments, a scorecard is generated for each participant that conveys both the quantity and quality of work they provided. A score can be based on a variety of factors, including, but not limited to, a percentage of users that cite to data vetted by the participant, a rating of usefulness (e.g., determined by surveys or other user feedback, measured user outcomes, reliance/stability over time, tested outcomes, audits, etc.), a deduction for errors found, a boost (e.g., 50%, 100%, 200%, etc.) for top performance within a peer group (e.g., top 5% curator, top 5 curator, etc.), a reduction (e.g., 10%, 20%, 50%, etc.) for low performance within a peer group (e.g., bottom 5%, 5 most mistake prone curators, etc.), the number of responses source material was cited, in response to end-user prompts, and the nature of subject matter domain or account in which the work was performed (e.g., paid accounts, free accounts, high value accounts, high liability risk accounts, etc.).

In some embodiments, curation system participants are given feedback (e.g., reports) that provide regular (e.g., monthly, weekly, daily, real-time) feedback on the factors that are evaluated in determining their share of the revenue. In some embodiments, the feedback includes a list of errors or important affirmative actions required (e.g., removing out-of-date information) to improve the system. In some embodiments, distribution of payment is contingent on correction of errors and/or completion of required affirmative actions.

In some embodiments, the systems and methods utilize an audit process to ensure the participants receiving a reward for participation are not gaming the system to maximize rewards without provide a commensurate contribution. In some embodiments, the auditing comprises comparing one or more performance parameters, that are not included in the reward calculation system, to confirm that the participant's contributions merited the associated reward. In some embodiments, the auditing comprises a review of one or more individual factors that are employed in the reward calculation system, to confirm the merit of the reward. The audit can include automated and/or human evaluations. In some embodiments, top performers are audited more frequently than other participants. In some embodiments, participants that have significant increases in earned rewards over two or more time periods, independent of the scale of the reward, are more frequently audited than other participants.

In some embodiments, the systems and methods employ a tracking system that tracks all user (e.g., curator, administrator, end user, etc.) metadata. User metadata includes, but is not limited to, username, time, location, device, documents, files, filenames, URLs, interactions, and all other implicit and explicit data related to curator activity. In some embodiments, the data is stored in one or more databases. In some embodiments, the tracking system generates a comprehensive and auditable log of all curator contributions and interactions to inform the refinement of the AI/ML system and subsequent citations, attributions, and revenue distributions. In some embodiments, the tracking system collects and/or stores information about curation system participants, including, but not limited to, username, role, date/time of login, location of login (e.g., IP address), device of login, source material submitted, reviewed, nominated, rated, and/or rejected, and interactions of users with data, and metadata about the context of the interaction. In some embodiments, some or all of this participant information (e.g., personal information) is not directly used in adjusting relevance scores, weighting relevance calculations, or retraining or refinement of the AI/ML system. In some embodiments, participants can select privacy settings that control the amount and nature of participant information that is stored or utilized.

In some embodiments, the system implements a specialized metadata collection and processing system focused particularly on training data source materials typical of published research and scientific journals, which are often in PDF format. In some embodiments, the metadata collection system extracts and stores multiple categories of metadata that enhance the verification and traceability of information, including: publication name and journal-impact-factor metrics, which provide quantifiable indicators of publication credibility; author information and associated h-index values, which serve as quantitative measures of author scholarly impact; precise publication date information, which permits chronological assessment of information currency; and standardized identifiers such as PubMed ID or DOI, which facilitate programmatic citation generation and source verification. This structured metadata architecture permits the system to generate machine-verifiable citations for each response produced by the AI system, which is particularly valuable for applications in scientific, healthcare, and medical domains where source verification is important.

In some embodiments, the metadata extraction subsystem employs multiple retrieval pathways. When source documents are imported through integration with established document databases such as PubMed or DOI.org, the system automatically queries and retrieves associated metadata through standardized APIs. In cases where complete metadata cannot be programmatically obtained, such as when a curator directly uploads a PDF document, the system employs a dedicated language model specifically configured for metadata extraction. This extraction model processes the textual content, document structure, headers, footers, and reference sections of the uploaded document to derive the required metadata parameters. The extraction process employs specialized prompting techniques optimized for academic and scientific document structures.

The collection and analysis of such data facilitates refinement of AI systems. For example, if the date of the most recent login of an expert on a particular topic (e.g., prostate cancer) was one year old, then a flag is sent to an oversight individual or group to determine whether certain analyses should or should not be performed by the system (e.g., providing information about the difficult choice between getting surgery, medicine or adopting a “wait and see” to slow-growing prostate cancer). In some embodiments, such time gap flags set a default “do not provide decision support” setting that prevents use of the system for particular purposes unless overridden by an appropriately senior supervisor. In some embodiments, for curators whose cumulative metadata indicate a high “rating” of quality of contributions and have demonstrated a regular engagement, their subsequent contributions would have an algorithmically calculated higher weighted impact on adjusting relevance scores, weighting relevance calculations, or otherwise fine tuning of the system for any errors they correct or training data than curators who have a lower “rating.” In some embodiments, for data that often yields high engagement (“clicks,” “views,” “further prompts”), the system initiates an additional chain of expert reinforcement so that these higher impact requests undergo more thorough vetting. In some such embodiments, the system is adjusted to suggest high engagement content more often, which creates a virtuous cycle.

In some embodiments, the systems and methods comprise a blind spot detection component that identifies gaps in the quality and/or quantity of data (e.g., questions from users that are not supported by sufficient curated research) and reports gaps to curators. Utilization of a blind spot detection component, by routing gap information into the expert curation process and incentivizing curators to find information that reduces or eliminates the gap, increases system accuracy and associated effectiveness and trust.

In some embodiments, the systems and methods comprise an interactive artificial intelligence language system interface, where users in a range of designated roles (e.g., “administrators”, “curators” and “customers”) can enter prompts, ask questions, provide individual information (e.g., for health care indications: age, race, gender, location, existing conditions, genomics, medications, biomarkers, etc.) receive answers, and rate responses from the AI language system trained on and generated from the cumulative “source” data entered by “curators”, and personalized according to information provided by users (e.g., customers) and the context of previous prompts, answers provided information. In some embodiments, answers are accompanied by citations and metadata illustrating the “sources” and “curators” whose training data inputs inform the AI language systems' response to a given prompt.

In some embodiments, individual user information, prompts, and responses provided by users while interacting with AI are used to tailor responses unique to their experience, and are not used to train the system writ large. In some embodiments, anonymized metadata surrounding their interactions, such as the content and structure of their prompts, and ratings of responses are incorporated into calculations to inform research, refinement, and optimization of the system, but only in aggregate and under the supervision of appropriate authorized personnel (e.g., administrators) within the curation system.

In some embodiments, the system generates and displays a flowchart or other information summary showing all contemplated information inputs and outputs for each type of participant that interacts with the system. For example, for an administrator, the system identifies the ranges of information inputs they will query the system with and the ranges of information that will be received from the system. The process is repeated for a curator. The process is repeated for an exemplary end user (e.g., customer). This may be the first time that the end user is exerting an influence on the system-making it important to identify and list their inputs and outputs. The process may be repeated for a number of exemplary individual users.

In some embodiments, the systems and methods comprise a two-stage diagnostic reinforcement learning system. In some embodiments, the two-stage diagnostic reinforcement learning system allows and encourages curation system participants to consistently test, rate, correct, and refine responses, and update, remove, or refine underlying source material AI training data they ingest into the system. In some embodiments, this module is designed to make participants responsible for the life cycle of the source material they input into the AI training database by creating a circular, continuous “rinse and repeat” feedback loop that keeps the source material accurate and contemporary.

In some embodiments, in addition to the automated quality control that the system provides, the curation process includes an iterative testing, evaluation, and annotation cycle which results in consistent, high-quality outputs when the given topic is activated during a query. After uploading source materials, providing curator annotations, or editing scoring rubrics, the system re-processes the document/annotation indexes, which allow immediate ad-hoc testing. Curators can submit questions for the system to answer, similar to how it would function in its final production form. An answer is generated using only sources from the topic being curated. In addition to generating an answer and citations, the system also provides the document chunks and annotations used, and the resulting scores from automated quality control steps.

In some embodiments, the system is configured to generate likely reasons for low scores. For example, the system may identify insufficient source material or applicable source material not present or not found. The system may also suggest remedial actions. For example, when applicable source material could not be found, the system may suggest either uploading additional source material, or providing annotations where an answer might have been derived.

In some embodiments, once the results of an ad-hoc test meet predetermined quality thresholds, a curator can create a formal evaluation. Evaluations provide a measure of the quality of answers over a statistically significant quantity of generated answers. The quantitative results of evaluations are also used as a tool to measure improvements to the system relative to previous evaluation scores. In some embodiments, evaluations can be scored automatically, manually, or through a combination of both approaches. Based on scores produced during evaluations, the system can suggest likely reasons for low scores or low consistency, and generate improvement suggestions. Suggestions may include changes to standard LLM tuning parameters, such as temperature, top_p, and other parameters, or proprietary tuning parameters, such as the weighting of general context versus personalized context. In embodiments where evaluations are scored automatically, the system can run multiple self-tuning cycles to arrive at optimal settings.

In some embodiments, when generating output from a topic, the system provides citations of source material. In some embodiments, the mechanism of providing citations leverages features of existing LLMs. An automated quality control, or evaluation system, can score the output in terms of correctness, adherence to instructions and sources, completeness, and consistency. In addition, the system may add a topic-specific, expert-in-the-loop scoring rubric. For example, for the topic of “healthspan via musculoskeletal health,” a curator-defined scoring rubric might include additional scores around the applicability of answers to an individual's specific musculoskeletal conditions. Generated output might be scored against checks such as “Do answers account for previous injuries? Do answers account for bone density?” and similar topic-specific considerations that would not be generally applicable to all topics.

In some embodiments, by incorporating this automated quality control into the curator workflow, the system can further identify potential gaps in the curated source material. Gaps may be addressed through additional annotation or through additional source material. Additionally, the system can perform automated quality control at the time of generation. The quality control results may be used to regenerate output or portions of output until a sufficiently high-quality response is provided. In some embodiments, when quality fails to pass checks, the system might instead explain that no answer can be given rather than providing potentially inaccurate information.

Use of such a system is exemplified by reference to a specific topical example: Lyme disease. Lyme disease is caused by a bacteria, Borrelia burgdorferi which is carried by black-legged ticks that feed on the blood of animals and humans. These ticks may feed on the blood of mice and chipmunks and become infected with the bacteria. The ticks may also feed on animals with larger territories such as deer, birds, dogs, and cats that can move the infected tick across very long distances to reach and infect new people. Participants may interact directly or indirectly with the system in several ways: (1) Top-down from experts curators who are vertical experts in Lyme disease and Rheumatology related to treatment for arthritis and chronic pain; (2) “Sideways” in cross-cutting areas like Entomology, Infectious diseases, and “One Health” epidemiologists that track the migration of ticks and the deer, birds, skunks, foxes, and other animals that carry them; and (3) Bottom-up from thousands to millions of recommendations of family home treatments, salves, poultices, vitamins, out-of-favor preventive medicine, or niche treatments that the AI can sort and present to the board of medical experts to adjudicate.

In some embodiments, where privacy is required or desired, the systems and methods comprise components to de-identified individual user data. In some embodiments, the de-identification process employs 3rd party mechanisms (e.g., via connected health devices, documents, etc.). In some embodiments, these channels can also be included in revenue sharing calculation. In some embodiments, users opt-in to share their anonymized data to evaluate the efficacy of recommendations and contribute results to future research, which can then funnel back into the system as a novel training data source. In some embodiments, users are compensated with revenue, free usage, recognition in the research, or by other mechanisms (e.g., virtual points that are redeemed to unlock more advanced features or for products, goods, or services).

Uses of the systems and methods described herein prevents, corrects, and reduces the impact of common sources of misinformation and hallucinations, including, contamination from LLMs trained on the wider Internet, unverified sources with no audit trail, outdated source material from AIs whose training data is updated at long intervals, “poison” or “trickery” or “jailbreaking” from bad actors, trolls, or hackers attempting to fool or compromise the LLM with malicious intents and/or prompts, and compromised “rinse and repeat’ cycles. The systems and methods also reduce or eliminate the problems of combining information improperly from multiple sources (which individually may be accurate), overfitting (not being able to adapt to new situations, and overly recommending what it has seen before without understanding differences in the situation), and input bias (e.g., source data may be accurate, but leaning towards certain perspectives) and the potentially associated compounding of such biases by training.

The systems and methods find use with a variety of AI/ML systems including, but not limited to, neural networks (e.g., with reinforcement learning through human feedback), large language models (LLMs) or mall language models (SLMs), large sequence models (e.g., where a sequence is defined as “diagnosis” defined as any of a set of determinations about the causality and each diagnosis has an associated regression model stored separately), fine-tuned large language models, an ensemble of machine learning models of any permutation of the above, and causal inference models (e.g., where interventions are modeled as causal and have associated effect coefficients; where interventions are modeled as causal and associated effect coefficients are determined through a series of prompts passed to any of the above models), Retrieval augmented generation (RAG) techniques (e.g., where prompts and information generated by LLMs is combined with data retrieved directly from data bases and other data sources), Model Context Protocol (MCP) systems (e.g., wherein a universal framework connects tools or other AI/ML models and systems and data sources of diverse contexts while emphasizing security), Agentic AI (e.g., where a system comprised of multiple components work together to autonomously set goals, create action plans, and perform workflows towards achieving them), and Mixture of Experts (MOE) techniques (e.g., where multiple smaller models trained to have expertise in specific fields are employed over a single monolithic model, a gating network is used to route inputs to the expert models and weigh their responses, and a collaboration/answer model is used to combine outputs into a single cohesive response). In some embodiments, the systems and methods utilize existing, deployed research and commercial systems including, but not limited to, OpenAI, ChatGPT, Hugging Face, Google Gemini, DeepMind, Med-PaLM, Mistral, Databricks, Anthropic, and/or Adept.

In some embodiments, the system leverages specific commercial AI components, including commercial LLMs such as Anthropic Claude, provided either directly or via cloud providers such as AWS; vector embedding models such as Cohere; vector search systems such as OpenSearch; OCR and data extraction systems such as Textract; and LLM data frameworks providing tools such as Llama-index semantic splitting.

In some embodiments, the system builds upon specific AI techniques, including but not limited to: Retrieval augmented generation (RAG); semantic similarity matching; in-context learning; chain of thought inference pipelines; one-shot and multi-shot prompting; and prompt engineering. These techniques, when combined with the expert curation systems and methods described herein, enhance the accuracy, reliability, and trustworthiness of the generated outputs.

In some embodiments, the systems and methods find use in the enhancement of healthspan. Numerous factors are involved in improving and maximizing healthspan.

Experiments conducted during the development of embodiments of the present technologies identified the most relevant factors that most significantly contribute to healthspan. While some individual factors were expected, some were not, leading to an unexpected priority list. The top identified worldwide factors were: atherosclerosis (and related heart attacks and strokes), cancer, neurodegenerative diseases (e.g., Alzheimer's disease, Parkinsons disease, dementia), infectious disease (e.g., tuberculosis, pneumonia, Sars-Cov2, antibiotic resistance, superbugs, etc.), metabolic syndrome (e.g., obesity, diabetes), sarcopenia/orthopedic (e.g., muscle wasting, hip fractures, disabling joint disease and pain), violence (e.g., accidents (e.g., cars, sports), gun violence, etc.), lower respiratory disease (e.g., COPD, bronchiectasis, asthma), despair (e.g., situational depression, hopelessness, self-medication, addiction, suicide, homicide), maternal morbidity and mortality, menopause, testosterone imbalances, kidney disease (e.g., end stage renal disease), liver disease (e.g., cirrhosis), accidents and injuries, and place factors (e.g., climate change (e.g., antecedent causes, subsequent effects), vector-borne, super storms, floods, migration, drought, crop failures, water-borne disease, air pollution/soot, plastics, ocean warming, loss of fish, species extinctions). The results included surprising findings, such as the significantly greater impact on lifespan related to relatively innocuous-sounding conditions (e.g., broken hips, new blindness due to untreated cataracts, postponing menopause in women) compared to treatments for cancer.

Notably, a number of these factors involve “cross-talk” in that there is some known and likely vast amounts of unknown correlation between them in terms of their impact on healthspan. The systems and methods provided herein, when evaluating these factors individually or in various combinations (e.g., evaluation of all factors), provides significant insight into actionable steps to enhance healthspan.

The systems and methods may further evaluate one or more individual risk factors. Some risk factors are relatively easy to obtain from health records (e.g., electronic medical records) or simple surveys. Such factors include, but are not limited to, age, gender at birth, race, health record data (e.g., clinical history and physical examination, prior illness and treatments, blood tests, prescribed medications, exercise supplements, wearables, etc.), social determinants of health (e.g., class, wealth, education, family background, etc.), geographic location by year and duration. Other risk factors may be determined by laboratory tests, connected devices or other sources, including, but not limited to “omics” (e.g., genomics, epigenomics, proteomics, microbiomics (e.g., gut, lung, oral-pharynx, vaginal, etc.)), “digital biomarkers” (e.g., data collected phones, cameras, voice recordings, wearables, sleep data, etc.), and “exposome” (e.g., place history (e.g., zip codes) over time related to air pollution, proximity to toxins, lead level, noise, etc.).

Assessment of these and other data allow the systems and methods of the present invention to identify both population-based, group-based, and individual-based interventions to improve healthspan. For example, use of the systems and methods identify correlations (e.g., use of hearing aids and/or better oral hygiene to reduce the impact or onset of Alzheimer's disease or dementia) that have relatively simple actionable steps to improve health, well-being, and longevity.

In some embodiments, when the system receives a healthspan query, it uses a variety of techniques, such as semantic similarity, to generate a relevance score between the query and each topic within the system. Each topic represents a collection of source documents, curator annotations, guides for which documents should be used and when/how, scoring rubrics, and related information. To produce a score on a single topic, the query is encoded as an embedding vector using an embedding model. The distance between the query vector and each chunk vector in the topic's RAG index is calculated using a vector database. The search returns the top “nearest” chunks along with their distances, and a relevance score is calculated using these distances. In some embodiments, where queries include personal details such as personal health information, geographic place factors, or other individual characteristics, those details are also encoded as vectors. Thus, answers to the healthspan queries may be personalized to an individual based on personalized health information, e.g., electronic health record, digital twins, etc., as well as population, community, and cohort data related to the individual.

In some embodiments, topics with high relevance scores are selected by the system as eligible to answer the query. For each eligible topic, the system retrieves top document chunks from the topic's associated RAG index. Using a similar vector index comparison, the system retrieves additional annotation data associated with the topic. Using similar vector comparisons, the system retrieves context relevant to personal information provided. This is performed as a separate step to ensure that both general information and personal information are incorporated into the retrieval of context. In some embodiments, the system creates a prompt payload using proprietary prompt engineering which incorporates the original query with relevant document chunks and annotation data. The prompt payload is sent to an LLM such as Anthropic Claude, Open AI ChatGPT, Google Gemini, or other suitable models. Citations are retrieved from the respective LLM system.

In some embodiments, each answer is checked against an automated quality control, which includes checks for accuracy against source material, completeness in answering the query, and consistency within the response. The quality control may include topic-specific scoring rubrics developed by the curation experts. Low scores may result in a full or partial regeneration of the answer. Depending on the scores across multiple topics, a single answer may be provided, or multiple perspectives may be presented. Citations are provided alongside the answer, permitting a human to trace through the applicable sources and reach an informed independent conclusion on the quality of the answer.

Understanding the relevant risk factors and contributors to healthspan provides insight into the characteristics of the personnel involved in the curation systems and methods. For example, the personnel, collectively, should have expertise, wisdom, and common sense in the following areas: preventative medicine, actuarial and longevity sciences, biostatistics, epidemiology (e.g., clinical, infectious disease, chronic disease), genomics, pharmacology, medical care, mental health, public health, health economics, health insurance, health planning, health behavior, environmental health, and climate science. Supporting expertise includes, but is not limited to, coaches, trainers, and therapists in the fields of nutrition, physical training, physical therapy, health-seeking behavior, life doulas, massage therapy, herbalism, integrative medicine, home health care, and mobile/traveling health care.

The analysis of multiple topics and sub-topics together prevents the systems for generating bad judgments that might be considered good judgment when viewed from the perspective of a single dimension. For example, based on information generated by a single-dimensional analysis, a functional medicine doctor might suggest estrogen replacement therapy to postpone the onset of menopause in a woman because data shows one year of postponement is associated with three more years of healthy life. However, an OB-GYN doctor might suggest if the woman has a certain genomic picture, that estrogen would increase her risk of breast or ovarian cancer. By evaluating information in multiple dimensions, with the associated expertise, adjudication of this legitimate debate would be elevated to an appropriate supervisory panel. Also, for example, climate expertise and infectious disease expertise, working together, provides a better evaluation, and if needed adjudication, of risks associated with malaria due to climate change.

In this context, the systems and methods provide a hybrid AI/expert decision support system. The people whose decisions are supported include individual health seekers, physicians and clinicians of many types (e.g., not just MD's but many forms of practitioners: pharmacists, nurses, dentists, community health workers, physical therapists, sports medicine specialists, etc.), hospital operators, health system executives, health insurance companies, governments, enterprises, and countries seeking to equitably and efficiently make resource allocation decisions. The expert support comes not only from providing highly valid information to expert users, but also the ability to access the experts within the curation systems and methods in adjudication processes.

Aggregation of data from large numbers of users allows for better decision-making and more efficient delivery of health services throughout the health care, preventive medicine, and public health systems of any enterprise or jurisdiction where it is used. For example, federal insurance programs, such as Medicare and Medicaid as well as private health and life insurers as well as HMOs have a difficult job predicting disease patterns (e.g., how many patients over 65 living in Mississippi or by the bayous of Louisiana are likely to have malaria and need specialized treatment five years from now or how will changes in the onset dates menopause affect the number of broken hips likely to emerge in their patient or covered populations-which also determines how many MRI and CT scanning machines they need to buy and how many radiologists they will have to have on staff, etc.).

At scale, the experience of users using the systems and methods provided herein creates nations of health seekers armed with the best data available and prevention in their hands (e.g., on the mobile devices) and in their communities to create advocates for better health policy and better use in the first instance of the huge amount of money spent by our governments and healthcare systems. Use of the systems and methods shifts the ratio of expenditures in favor of preventive medicine over curative medicine.

Likewise, the systems and methods provide highly individualized, proactive guidance and prevention integrated into the workflow of clinicians and other healthcare workers who can readily leverage this (e.g., via in-person, virtual and digitally enabled hybrid care) to personalize health optimization (i.e., “precision wellness”), to improve current and future function and healthspan, as well as to best screen patients most appropriately based on insights of their specific patient's relative risk (e.g., colonoscopy screening could be indicated before or after the general age 45 guidelines of today). This facilitates highly personalized proactive and preventative care plans (e.g., that adapt over time based on new data/insights). By considering factors such as genetic predispositions, biomarker profiles (e.g., both lab data and data from wearables) and trends, and treatment response data, the system can recommend tailored preventative or therapeutic medication and/or evidence-based supplement regimens, dosage adjustments, and diet and lifestyle interventions. This lengthens healthspan and lowers downstream medical costs for acute and chronic disease. Further, the system can communicate the guidance to the individual users through modalities and platforms highly attuned to the user's age, culture, language, personality, etc, offer individually tailored insights, recommendations, goals, and progress. The system can act as an “Agent” or “Agentic AI” and seamlessly provide relevant information, products, services, professionals, and resources, and perform tasks to support users on their journey to day to day wellness, increased healthspan. Interactions with the system can occur across a range of devices, platforms, settings, and technologies (web portals, mobile phones, smart watches, connected homes, Internet of Things (IOT), and others) ensuring a seamless and continuous experience with users wherever they go and throughout their lives.

### EXAMPLES

### Example 1

### Atherosclerosis Curation System

A table of an exemplary tiered provisional system for authenticated individuals to select, upload, ingest, edit, and update content for atherosclerosis is provided in Table 1. Atherosclerosis (sometimes referred to as hardening of the arteries) is a precursor to atheromas, which obstruct blood flow distal to the lesion, leading to large numbers of both strokes (if the atheroma is in the blood vessels serving the brain) and heart attacks (if in the coronary arteries). The tiered provisional system includes: a medical adjudication board comprised of medical experts; a specialized medical advisory board including board certified subject matter experts; one or more super administrators; one or more administrators; one or more super curators; one or more curators; one or more commentators; one or more moderators; and a plurality of end users. Each of the individuals in the tiered systems can carry on the roles and responsibilities of the indicated tier or any tier under the indicated tier. For example, an associate professor of neurology may fulfill the roles or contribute to the responsibilities of a curator or super curator. Internal medicine is a superset of both neurology (strokes) and cardiology (heart attacks) and thus would be more appropriate in this instance.

TABLE 1

| Role | Responsibilities | Credentials |
|---|---|---|
| Medical | Acts as controlling body to adjudicate | Vetted medical experts at the |
| Adjudication | when one specialty differs in | top of fields including an |
| Board | recommendations from another (e.g., | adjacent to atherosclerosis, |
| | when orthopedic doctors recommend rest | such as: former directors of |
| | for back injuries but the cardiologist | government health agencies |
| | recommends exercise) | (e.g., Centers for Disease |
| | Names (e.g., nominates, invites, votes | Control and Prevention, |
| | for, approves), authenticates, and revokes | National Institutes of Health, |
| | Specialized Medical Advisory Board | Food and Drug |
| | Members | Administration) and non- |
| | Edits responsibilities of Specialized | government scientific |
| | Medical Advisory Board Members | organizations (e.g., National |
| | Defines, provisions, and creates discrete | Academy of Medicine) |
| | atherosclerosis databases or knowledge | |
| | bases (e.g., databases or knowledge bases | |
| | accessible by a generative AI inference | |
| | system, curated knowledge bases, | |
| | language model training databases, etc.) | |
| Specialized | Names (e.g., nominates, invites, votes | Established board-certified |
| Medical | for, approves), authenticates, and revokes | MDs, MPHs, PhDs, and |
| Advisory | Super Administrators | subject matter experts (e.g., |
| Board | Edits responsibilities of Super | chairpersons of leading |
| | Administrators | research institutions) |
| Super | Defines, provisions, and audits discrete | Leading researchers or |
| Administrator | atherosclerosis databases or knowledge | physicians (PhD, MD, |
| | bases (e.g., databases or knowledge bases | Published Author) in medical |
| | accessible by a generative AI inference | fields associated with |
| | system, curated knowledge bases, | atherosclerosis (e.g., Deans |
| | language model training databases, etc.) | of Neurology, Cardiology, |
| | Names (e.g., nominates, invites, votes | Lipid Research at Top 5 US |
| | for, approves), authenticates, and revokes | Medical School |
| | Administrators | |
| | Edits responsibilities of Administrators | |
| | All Administrator functionalities | |
| Administrator | Ability to audit atherosclerosis databases | Professors (PhD, MD) in |
| | or knowledge bases (e.g., databases or | medical fields (e.g., |
| | knowledge bases accessible by a | Neurology, Cardiology, Lipid |
| | generative AI inference system, curated | Research) associated with |
| | knowledge bases, language model | atherosclerosis at Top 5 US |
| | training databases, etc.) to which they | Medical School |
| | have access | |
| | Names (e.g., nominates, invites, votes | |
| | for, approves), authenticates, and revokes | |
| | Super Curators | |
| | Edits responsibilities of Super Curators. | |
| | All Super Curator functionalities | |
| Super Curator | Audits, selects, ingests, updates, and | Professors (PhD, MD) in |
| | removes training data from | medical fields (e.g., |
| | atherosclerosis training databases to | Neurology, Cardiology, Lipid |
| | which they have access | Research) associated with |
| | Reviews all recommendations, ratings, | atherosclerosis at Leading |
| | responses, flags, and comments from all | Medical Schools and |
| | roles | Institutions |
| | Names (e.g., nominates, invites, votes | |
| | for, approves), authenticates, and revokes | |
| | Curators | |
| | Edits responsibilities of Curators | |
| | All Curator functionalities | |
| Curator | Audits, reviews, flags, recommends, and | Physicians and professors |
| | rates source materials, prompts, and | (PhD, MD) in medical fields |
| | responses for authorized training | (e.g., Neurology, Cardiology, |
| | databases | Lipid Research) associated |
| | Names (e.g., nominates, invites, votes | with atherosclerosis |
| | for, approves), authenticates, and revokes | |
| | Commentators | |
| | Edits responsibilities of Commentators | |
| Commentator | Reviews, recommends, and rates source | Graduate Students (PhD, |
| | materials, prompts, and responses for | MD) in medical fields (e.g., |
| | authorized training databases | Neurology, Cardiology, Lipid |
| | Names (e.g., nominates, invites, votes | Research) associated with |
| | for, approves), authenticates, and revokes | atherosclerosis |
| | Moderators | |
| | Edits responsibilities of Moderators | |
| Moderator | Reviews end-user prompts, responses, | Graduate students or degree |
| | flags, ratings, and recommendations | holders in any medical field |
| | Names (e.g., nominates, invites, votes | (PhD, MPH, MD, etc.) |
| | for, approves), authenticates, and revokes | |
| | End Users | |
| End User | Answers and provides suggested edits or | Doctors, Professors, |
| | new survey questions | Graduate Students, Nurses, |
| | Enters prompts and questions and | Researchers, Patients, Health |
| | rates/comments on responses and | Seekers, Health Providers |
| | recommendations to prompts and | |
| | questions | |
| | Connects and Integrates 3rd party health | |
| | data (from connected devices such as | |
| | Fitbit, Aura, Apple Health, 23 & me3, and | |
| | personal patient records) | |
| | Accesses a dashboard of personalized | |
| | health metrics, trends, recommendations, | |
| | resources, products, services, | |
| | professionals, community discussions, | |
| | and the like | |
| Evaluator/Tester | Tests and evaluates exemplary or | |
| | anticipated End User prompts and | |
| | responses to ensure quality, accuracy, | |
| | and source attribution | |
| | Experiments with prompt engineering to | |
| | identify quality improvements in | |
| | responses and outputs, including those | |
| | identified by other user Roles | |
| | Documents, flags, and communicates | |
| | errors or quality variance to Data Quality | |
| | Control Team | |

### Example 2

U.S. Case Law Curation System

| Role | Responsibilities | Credentials |
|---|---|---|
| Legal | Acts as controlling body to adjudicate | Vetted and distinguished |
| Adjudication | when one specialty or legal interpretation | experts at the top of US legal |
| Board | differs in recommendations from another | fields (e.g. former local, state |
| | (e.g., when some case law judgments | and federal judges and non- |
| | may overlap, conflict or have different | government legal and |
| | jurisdictions) | academic organizations (e.g., |
| | Defines, provisions, and creates discrete | Bar Association, Deans of |
| | US Case Law databases or knowledge | Top 10 US Law Schools, |
| | bases (e.g., databases or knowledge bases | Non Partisan Not-for-Profit |
| | accessible by a generative AI inference | Organizations such as |
| | system, curated knowledge bases, | ACLU) |
| | language model training databases, etc.) | |
| | and ontology | |
| | Names (e.g., nominates, invites, votes | |
| | for, approves), authenticates, and revokes | |
| | Specialized Legal Advisory Board | |
| | Members | |
| | Edits responsibilities of Specialized | |
| | Legal Advisory Board Members | |
| Specialized | Names (e.g., nominates, invites, votes | Leading researchers, authors, |
| Legal | for, approves), authenticates, and revokes | legal scholars, and former |
| Advisory | Super Administrators | practicing attorneys in US |
| Board | Edits responsibilities of Super | Case Law across a section of |
| | Administrators | legal fields and jurisdictions |
| | | (e.g., intellectual property, |
| | | litigation at state and federal |
| | | levels) |
| Super | Defines, provisions, and audits discrete | Distinguished bar-certified |
| Administrator | US Case Law databases or knowledge | JDs and specialized US Case |
| | bases (e.g., databases or knowledge bases | Law subject matter expertise |
| | accessible by a generative AI inference | across all US jurisdictions |
| | system, curated knowledge bases, | (across 50 US states and |
| | language model training databases, etc.) | federal) in a cross-section of |
| | Names (e.g., nominates, invites, votes | specialized US Case Law |
| | for, approves), authenticates, and revokes | fields (such as intellectual |
| | Administrators | property and litigation, etc.) |
| | Edits responsibilities of Administrators | with 15 years of experience |
| | All Administrator functionalities | in US Case Law (e.g., Deans |
| | | of Top 25 Law Schools) |
| Administrator | Ability to audit US Case Law databases | Distinguished Professors in |
| | or knowledge bases (e.g., databases or | US Case Law from Top 10 |
| | knowledge bases accessible by a | US Law Schools in each state |
| | generative AI inference system, curated | with 15 years of experience |
| | knowledge bases, language model | in US Case Law |
| | training databases, etc.) to which they | |
| | have access | |
| | Names (e.g., nominates, invites, votes | |
| | for, approves), authenticates, and revokes | |
| | Super Curators | |
| | Edits responsibilities of Super Curators | |
| | All Super Curator functionalities | |
| Super Curator | Audits, selects, ingests, updates, and | Professors and Bar-Certified |
| | removes training data from | Graduates (JD) from Top 5 |
| | atherosclerosis training databases to | law schools in each US state |
| | which they have been given access | with 10 years of experience |
| | Reviews all recommendations, ratings, | in US Case Law |
| | responses, flags, and comments from all | |
| | roles | |
| | Names (e.g., nominates, invites, votes | |
| | for, approves), authenticates, and revokes | |
| | Curators | |
| | Edits responsibilities of Curators | |
| | All Curator functionalities | |
| Curator | Audits, reviews, recommends, and rates | Bar-Certified Graduates (JD) |
| | source materials, prompts, and responses | from Top 10 law schools in |
| | for authorized training databases | each US state with 5 years of |
| | Names (e.g., nominates, invites, votes | experience in US Case Law |
| | for, approves), authenticates, and revokes | |
| | Commentators | |
| | Edits responsibilities of Commentators | |
| Commentator | Reviews, recommends, and rates source | Graduate law school |
| | materials, prompts, and responses for | students, nominated, vetted, |
| | authorized training databases | and approved by their |
| | Names (e.g., nominates, invites, votes | professors or peers with |
| | for, approves), authenticates, and revokes | Curator credentials or above |
| | Moderators | |
| | Edits responsibilities of Moderators | |
| Moderator | Reviews end-user prompts, responses, | Undergraduate legal students |
| | flags, ratings, and recommendations | or graduate in any legal field, |
| | Names (e.g., nominates, invites, votes | nominated, vetted, and |
| | for, approves), authenticates, and revokes | approved by their professors |
| | End Users | or Graduate Students who |
| | | have Commentator |
| | | credentials or above |
| End User | Enters prompts and questions and | Lawyers, Judges, Professors, |
| | rates/comments on responses, citations to | Students, Journalists, |
| | prompts and questions | Citizens, Legal Counsel for |
| | Answers and provides suggested edits or | Private Sector, Public Sector, |
| | new survey questions | and Not-for-Profit |
| | Accesses a dashboard of historical | Organizations |
| | prompts, responses, searches, and | |
| | relevant citations and source materials | |
| Evaluator/Tester | Tests and evaluates exemplary or | |
| | anticipated End User prompts and | |
| | responses to ensure quality, accuracy, and | |
| | source attribution | |
| | Experiments with prompt engineering to | |
| | identify quality improvements in | |
| | responses and outputs, including those | |
| | identified by other user Roles | |
| | Documents, flags, and communicates | |
| | errors or quality variance to Data Quality | |
| | Control Team | |

### Example 3

### Curation System Workflow

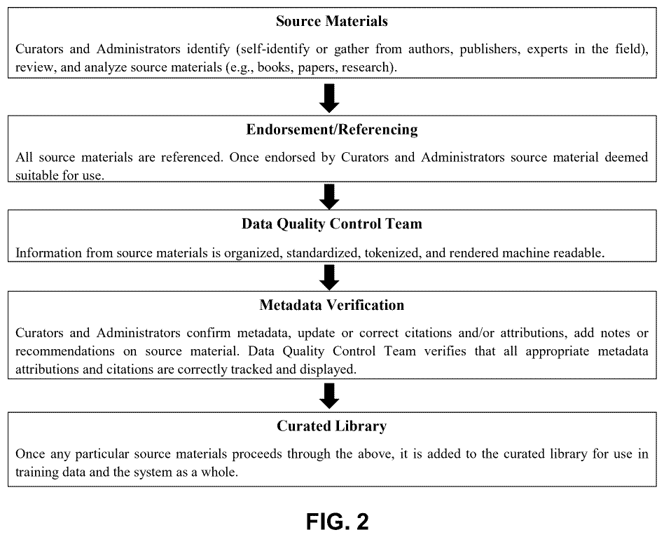

Curation systems for any topic or subject area include a group of individuals, as described in Examples 1 and 2, which contribute to the curation system workflow. As shown in FIG. 2, the curation systems provide a means to identify and curate source materials. Within the curation system, one of the members of the curation system would manage the curation of the library of source materials which is used to formulate the training databases and the AI system.

Any single source material, e.g., book, research paper, presentation, is identified, reviewed, and analyzed within the curation system by the administrators and curators. Each potential source material entering the curation system is referenced and once a quorum of endorsements from the administrators and curators is achieved the source material is sent to a Data Quality Control Team comprising data scientist and quality control engineers, optionally in conjunction with a system of language learning models, for organizing, standardizing, tokenizing, and casting the material in a machine-readable format. The metadata associated with the source materials as well as any citations or attributions to the source material is verified and, if needed, updated by a member of the curation team. Over time, the Data Quality Control Team verifies that all appropriate metadata attributions and citations are correctly tracked and displayed and that the changelog reflecting these updates and references all modifications, contributions, and attributions

Once the source material passes through each of the curation steps above, the source material is curated in the library alongside any other additional curated source materials for use in both the training data and the system as a whole. The administrators and curators confirm and verify that the source material is correctly applied to the AI systems.

### Example 4

### Source Materials

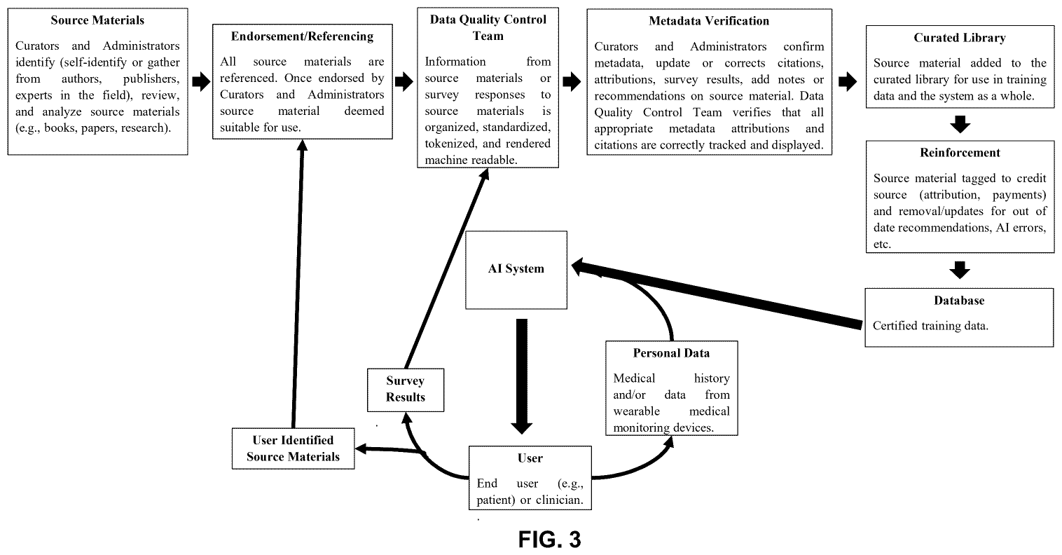

As described in Example 3, top-down discovery of a source material, e.g., medical journal publication and professional network of administrators and curators, enters the curation system following their identification by the administrators and curators. However, users of the AI system or other members of the curation team may generate a second, bottom-up, type of source material. This bottom-up source material may be generated through user interactions, prompts, responses, ratings, and comments. As shown in FIG. 3, this data can be fed into the curation system for referencing, and review and verification of the source material and metadata by administrators and curators prior to casting by the data scientists and quality control engineers into materials suitable for use in the training databases and the AI system.

Source materials can be annotated during, after, or concurrently with discovery and selection. The source materials can be annotated and tagged with metadata for a particular specialty category (e.g., cardiology, pediatrics, etc.), evidence level classification (e.g., RCT, observational study, expert consensus), publication date and currency indicators, target audience (e.g., clinicians vs. patients), geographical relevance, confidence/certainty level of medical claims. The source materials can be annotated during the curation to extract key technological features, e.g., conditions, treatments, symptoms, identify causal relationships between concepts, mark contraindications and warnings, highlight or tag process and related outcomes, e.g., diagnostic criteria, treatment protocols, and the resulting treatment outcome.

### Example 5

### Formatting and Categorizing Source Materials

The data from the source materials is organized into a relational database facilitating extraction of the key/value pairs into a flat file or by a dynamic schema management tool, such as HarperDB, MongoDB, Pyspark, CrateDB, and the like.

For example, for a journal article or manuscript from a peer-reviewed publication, the research is distilled into a summary by a generative AI agent using an LLM that includes general knowledge of English and terms used in health, medicine, biology, etc. The summary is structured in a standard, tokenized form. For example:

Title: Rotator cuff related shoulder pain: Assessment,
management and uncertainties.
Authors: Jeremy Lewis
Publication Date: 2016 Jun. 1
Journal: Manual therapy
Link: https://doi.org/10.1016/j.math.2016.03.009
Abstract: Rotator cuff related shoulder pain (RCRSP)
is an over-arching term that encompasses a spectrum of
shoulder conditions including subacromial pain
(impingement) syndrome, rotator cuff
tendinopathy, partial thickness rotator
(RC) tears and atraumatic full thickness rotators' tears.

Additional tagging may be applied to indicate if the source material contains information relevant to determinants, mediators, interventions, or outcomes. Additional tagging may indicate whether the source material might be specific to certain populations included in the study. Additional tagging may include curator notes, comments, observations, rationalizations, and ratings.

### Example 6

### Prompts and Questions

End users (e.g., professionals, students, researchers, service providers, service users, individuals associated with advocacy groups, government employees, and general individuals) interact with the system in a variety of ways, as described above. One aspect of an end user's experience is to be presented with survey questions during or after use as well as enter prompts and questions and rate and comment on responses and recommendations based on prompts and questions. The presentation of these questions as well as the user's prompts, questions, responses, and recommendations may use any form of media, including text-, audio-, video-based.

During use of the system, an end user may be prompted with questions to determine if they are having a good or poor experience. If an end user is having a poor experience, they may be connected to a system assistant to help navigate or integrate with the system and gather more information regarding the user's experience to troubleshoot or guide the user to their goal.

When an end user enters a question and receives a response, a survey may be generated by a survey instrument or rating system within the AI program to ask questions such as:
Did you find this answer satisfactory? (Rate on a scale of 1-10)
Does this answer pertain to you/your situation?
Are the actions suggested in this answer realistic or feasible for you/your situation?
Do you plan to implement this advice in your health-seeking behavior?
Did you find this advice outdated? if so can you suggest a newer/better answer and if so can you also suggest source data for that answer
Did you find the answer helpful but the data cited unpersuasive?
Do you have any comments (or questions) you would like sent to the expert group who imputed this data/answer?
Would you like to be connected to a group discussion with other people who have the same question or who received similar answers?
Would you like to be connected to local resources, professionals, and other products and services to further assist you taking action towards your goals?
Are you interested in participating in and contributing to new research on healthspan to improve the system and benefit other health seekers like you?

Users who provide beneficial details and answer can be rewarded (e.g., a free subscription or free products, etc.) for participating in the curation system. The survey or prompts may include questions such as:
We have a reward system for people who answer many of these feedback questions or would like to contribute to additional healthspan research. Would you like to know more about the reward and research programs?
   Members of the curation system (e.g., curators and administrators) are given regular (e.g., monthly) reports of these answers and required to “fix” any errors, and remove out-of-date info.

1.-35. (canceled)

36. A system for responding to healthspan queries, comprising a computer processor configured to: a) receive one or more healthspan queries; b) process said one or more healthspan queries with an artificial intelligence (AI) component that has been trained using expert curated source information from three or more topics selected from atherosclerosis, cancer, neurodegenerative diseases, infectious disease, metabolic syndrome, sarcopenia/orthopedic, violence, lower respiratory disease, despair, maternal morbidity and mortality, menopause, testosterone imbalances, kidney disease, liver disease, accidents and injuries, and geographic place factors, to generate one or more answers; and c) display said one or more answers to a user.

37. The system of claim 36, wherein the AI component has been trained using expert curated source information from five or more of said topics.

38. The system of claim 36, wherein the AI component has been trained using expert curated source information from ten or more of said topics.

39. The system of claim 36, wherein the AI component has been trained using expert curated source information from each of said topics.

40. The system of claim 36, wherein the three or more topics comprises geographic place factors.

41. The system of claim 40, wherein the geographic place factors comprise climate change information.

42. The system of claim 36, wherein the three or more topics comprises despair.

43. The system of claim 36, wherein the expert curated source information is generated using a system for expert curation of training data source materials for an artificial intelligence (AI) system comprising a computer processor that tracks a plurality of individuals wherein any or all of the plurality of individuals validate each source material for a given subject matter to generate a curated library of validated source materials for the given subject matter for use as training data for the AI system.

44. The system of claim 36, wherein the user is a health care worker.

45. The system of claim 36, wherein the user is a patient.

46. A method for responding to healthspan queries, comprising: receiving one or more healthspan queries, processing said one or more healthspan queries with a system of claim 36 to generate one or more answers, and displaying said one or more answers to a user.

47.-50. (canceled)
